SEQUENCE LISTING (44 sequences)

SEQ ID NO: 1 - DNA, 35 bp, synthetic construct
  source 1..35: mol_type = other DNA
gcaaagtgtacgacaagaacatgtgcagctttctg

SEQ ID NO: 2 - DNA, 35 bp, synthetic construct
  source 1..35: mol_type = other DNA
cagaaagctgcacatgttcttgtcgtacactttgc

SEQ ID NO: 3 - DNA, 21 bp, synthetic construct
  source 1..21: mol_type = other DNA
ggacagcagagatccagtttg

SEQ ID NO: 4 - DNA, 20 bp, synthetic construct
  source 1..20: mol_type = other DNA
ggtccgttccaggatcttct

SEQ ID NO: 5 - DNA, 20 bp, synthetic construct
  source 1..20: mol_type = other DNA
tccagtgtggtggaattctg

SEQ ID NO: 6 - DNA, 20 bp, synthetic construct
  source 1..20: mol_type = other DNA
tatcgctggggaactttctg

SEQ ID NO: 7 - DNA, 20 bp, synthetic construct
  source 1..20: mol_type = other DNA
tgctgcacaacatcccttac

SEQ ID NO: 8 - DNA, 20 bp, synthetic construct
  source 1..20: mol_type = other DNA
tgctggtttcgtccttcttt

SEQ ID NO: 9 - DNA, 20 bp, synthetic construct
  source 1..20: mol_type = other DNA
cctacaagcggaagaacacc

SEQ ID NO: 10 - DNA, 20 bp, synthetic construct
  source 1..20: mol_type = other DNA
gttcttgctgtcccagtggt

SEQ ID NO: 11 - DNA, 20 bp, synthetic construct
  source 1..20: mol_type = other DNA
ctgaagaaggatggcagctc

SEQ ID NO: 12 - DNA, 20 bp, synthetic construct
  source 1..20: mol_type = other DNA
cttgggtgggttactccaga

SEQ ID NO: 13 - RNA, 20 nt, synthetic construct
  source 1..20: mol_type = other RNA
agagagagcttggttaactt

SEQ ID NO: 14 - DNA, 25 bp, synthetic construct
  source 1..25: mol_type = other DNA
cactgtgtggtatccttattttgga

SEQ ID NO: 15 - DNA, 25 bp, synthetic construct
  source 1..25: mol_type = other DNA
aggtaaatttcctctgggtaatgga

SEQ ID NO: 16 - DNA, 55 bp, Homo sapiens
  source 1..55: mol_type = genomic DNA
tgtcttctccctttcacatactatttatgtacacgtcgaaagacaagttgaactt

SEQ ID NO: 17 - DNA, 38 bp, Homo sapiens
  source 1..38: mol_type = genomic DNA
tcgactgtcttctccctttcacatactatttagaactt

SEQ ID NO: 18 - DNA, 45 bp, Homo sapiens
  source 1..45: mol_type = genomic DNA
tcgactgtcttctccctttcacatactataagacaagttgaactt

SEQ ID NO: 19 - DNA, 56 bp, Homo sapiens
  source 1..56: mol_type = genomic DNA
ctgtcttctccctttcacatactatttatgtacacgtcgaaagacaagttgaactt

SEQ ID NO: 20 - DNA, 55 bp, Homo sapiens
  source 1..55: mol_type = genomic DNA
tcgactgtcttctccctttcacatactatttatgtacacgtcgaaagacaagttg

SEQ ID NO: 21 - DNA, 60 bp, Homo sapiens
  source 1..60: mol_type = genomic DNA
tcgactgtcttctccctttcacatactatttatgtacacgtcgaaagacaagttgaactt

SEQ ID NO: 22 - DNA, 54 bp, Homo sapiens
  source 1..54: mol_type = genomic DNA
tcgactgccctttcacatactatttatgtacacgtcgaaagacaagttgaactt

SEQ ID NO: 23 - DNA, 47 bp, Homo sapiens
  source 1..47: mol_type = genomic DNA
ccctttcacatactatttatgtacacgtcgaaagacaagttgaactt

SEQ ID NO: 24 - DNA, 60 bp, Homo sapiens
  source 1..60: mol_type = genomic DNA
tcgactgtcttctccctttcacatactatttttgtacacgtcgaaagacaagttgaactt

SEQ ID NO: 25 - DNA, 55 bp, Homo sapiens
  source 1..55: mol_type = genomic DNA
tcgactgtcttctccccatactatttttgtacacgtcgaaagacaagttgaactt

SEQ ID NO: 26 - DNA, 16 bp, Homo sapiens
  source 1..16: mol_type = genomic DNA
tcgactgtcttctccc

SEQ ID NO: 27 - DNA, 58 bp, Homo sapiens
  source 1..58: mol_type = genomic DNA
gactgtcttctccctttcacatactatttatgtacacgtcgaaagacaagttgaactt

SEQ ID NO: 28 - DNA, 28 bp, Homo sapiens
  source 1..28: mol_type = genomic DNA
tcgactgtcttctccccaagttgaactt

SEQ ID NO: 29 - DNA, 32 bp, Homo sapiens
  source 1..32: mol_type = genomic DNA
tcgactgtcttctcccaagacaagttgaactt

SEQ ID NO: 30 - DNA, 33 bp, Homo sapiens
  source 1..33: mol_type = genomic DNA
tcgactgtcttctcccaaagacaagttgaactt

SEQ ID NO: 31 - DNA, 51 bp, Homo sapiens
  source 1..51: mol_type = genomic DNA
tcgactgtcttctcccctatttatgtacacgtcgaaagacaagttgaactt

SEQ ID NO: 32 - DNA, 18 bp, Homo sapiens
  source 1..18: mol_type = genomic DNA
tcgactgtcttctccctt

SEQ ID NO: 33 - DNA, 39 bp, Homo sapiens
  source 1..39: mol_type = genomic DNA
tcgactgtcttctcccacgtcgaaagacaagttgaactt

SEQ ID NO: 34 - DNA, 15 bp, Homo sapiens
  source 1..15: mol_type = genomic DNA
tcgactgtcttctcc

SEQ ID NO: 35 - DNA, 59 bp, Homo sapiens
  source 1..59: mol_type = genomic DNA
agctgacagaagagggaaagtgtatgataaatacatgtgcagctttctgttcaacttga

SEQ ID NO: 36 - protein, 19 aa, Homo sapiens
ADRRGKVYDKYMCSFLFNL

SEQ ID NO: 37 - DNA, 57 bp, Homo sapiens
  source 1..57: mol_type = genomic DNA
gttgacctagatgagatgtcgttcacttttactgagctacagaaaaacagaaatcag

SEQ ID NO: 38 - DNA, 59 bp, Homo sapiens
  source 1..59: mol_type = genomic DNA
gttgacctagatgagatgtcgttcacttttactgagctacagaaaaacatagaaatcag

SEQ ID NO: 39 - DNA, 52 bp, Homo sapiens
  source 1..52: mol_type = genomic DNA
gttgactgagatgtcgttcacttttactgagctacagaaaaacagaaatcag

SEQ ID NO: 40 - DNA, 16 bp, Homo sapiens
  source 1..16: mol_type = genomic DNA
gttgaccagaaatcag

SEQ ID NO: 41 - DNA, 63 bp, Homo sapiens
  source 1..63: mol_type = genomic DNA
gttgacctagatgagatgtcgttcacttttactgagctacaggtttcttgtataaatataagc

SEQ ID NO: 42 - DNA, 50 bp, Homo sapiens
  source 1..50: mol_type = genomic DNA
gttgacctagatgagatgtcgttcacttttactgagctacagaaaaacgc

SEQ ID NO: 43 - DNA, 86 bp, Homo sapiens
  source 1..86: mol_type = genomic DNA
gttgacctagatgagatgtcgttcacttttactgagctacagaaaaacatagaaatcaggtaaagtttcttgtattaaatataagc

SEQ ID NO: 44 - protein, 20 aa, Homo sapiens
VDLDEMSFTFTELQKNIEIR

(19) United States
(12) Patent Application Publication
(10) Pub. No.: US 2025/0146080 A1
(43) Pub. Date: May 8, 2025

(54) METHODS FOR OVERCOMING TAZEMETOSTAT-RESISTANCE IN CANCER PATIENTS

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Yaniv Kazansky, New York, NY (US); Alex Kentsis, New York, NY (US)

(21) Appl. No.: 18/838,962

(22) PCT Filed: Feb. 16, 2023
(86) PCT No.: PCT/US23/62737

§ 371 Date: Aug. 15, 2024

Related U.S. Application Data
(60) Provisional application No. 63/311,241, filed on Feb. 17, 2022.

(51) Int. Cl. C12Q 1/6886; A61K 31/5377; A61K 45/06; G01N 33/574

(52) U.S. Cl. CPC C12Q 1/6886; A61K 31/5377; A61K 45/06; G01N 33/57407; C12Q 2600/106; C12Q 2600/158; G01N 2333/4703

(57) ABSTRACT

The present disclosure provides methods for overcoming tazemetostat resistance in patients diagnosed with or suffering from sarcomas. In some embodiments, the methods of the present technology include administering tazemetostat in combination with an ATR inhibitor, a CDK4/6 inhibitor or an AURK inhibitor.

### CROSS-REFERENCE TO RELATED APPLICATIONS

This application is the U.S. National Stage Application of International Application No. PCT/US2023/062737, filed Feb. 16, 2023, which claims the benefit of and priority to U.S. Provisional Patent Application No. 63/311,241, filed Feb. 17, 2022, the contents of each of which is incorporated herein by reference in its entirety.

### SEQUENCE LISTING

The instant application contains a Sequence Listing which has been submitted electronically in .xml format and is hereby incorporated by reference in its entirety. Said, .xml copy, created on Aug. 14, 2024, is named 115872-3066_SL.xml and is 40,960 bytes in size.

### TECHNICAL FIELD

The present technology relates generally to methods for overcoming tazemetostat resistance in patients diagnosed with or suffering from sarcomas.

### BACKGROUND

The following description of the background of the present technology is provided simply as an aid in understanding the present technology and is not admitted to describe or constitute prior art to the present technology.

Tazemetostat (EPZ-6438) is an EZH2 inhibitor recently approved for the treatment of SMARCB1-deficient sarcomas (i.e. malignant rhabdoid tumor and epithelioid sarcoma). Despite its recent approval, patient response rates to tazemetostat remain low at ˜15% (Gounder et al, 2021, www.epizyme.com/wp-content/uploads/2021/06/Cohort-5_6_Immune-Priming_ASCO_Poster_Final.pdf).

Accordingly, there is an urgent need to identify clinically actionable combination therapies that show synergy with tazemetostat, increasing patient response rates and overcoming intrinsic and acquired resistance.

### SUMMARY OF THE PRESENT TECHNOLOGY

In one aspect, the present disclosure provides a method for treating sarcoma in a subject in need thereof comprising administering to the subject an effective amount of tazemetostat and an effective amount of a CDK4/6 inhibitor. In some embodiments, the CDK4/6 inhibitor is selected from the group consisting of palbociclib, ribociclib, and abemaciclib.

In one aspect, the present disclosure provides a method for treating sarcoma in a subject in need thereof comprising administering to the subject an effective amount of tazemetostat and an effective amount of an AURK inhibitor. The AURK inhibitor may be a selective inhibitor of AURKB or a pan AURK inhibitor. Examples of AURK inhibitors include, but are not limited to tozasertib, SP-96, AT9283, danusertib (PHA-739358), AMG900, cenisertib, SNS-314, barasertib, hesperadin, AZD1152, GSK1070916, CYC116, BI 811283, AZD2811, PHA680632, reversine, CCT129202, CCT137690, S49076, PF-03814735, chiauranib, Alisertib, quercetin, and VX-680.

In another aspect, the present disclosure provides a method for treating sarcoma in a subject in need thereof comprising administering to the subject an effective amount of tazemetostat and an effective amount of an ATR inhibitor. Examples of ATR inhibitors include, but are not limited to elimusertib (BAY1895344), Schisandrin B, NU6027, dactolisib (NVP-BEZ235), EPT-46464, Torin 2, VE-821, AZ20, M4344 (VX-803), berzosertib (M6620 (VX-970)), and ceralasertib (AZD6738).

Additionally or alternatively, in some embodiments of the methods disclosed herein, the subject is non-responsive to at least one prior line of cancer therapy such as chemotherapy or immunotherapy. In certain embodiments, the subject is human.

In any and all embodiments of the methods disclosed herein, the sarcoma is a rhabdoid sarcoma or an epithelioid sarcoma. In some embodiments, the sarcoma comprises a deficiency in a BAF chromatin remodeling complex component or SMARCB1. Additionally or alternatively, in some embodiments, the subject comprises an acquired mutation in EZH2, CDKN2A/B, CDKN1A, ANKRD11 or RB1. In any and all embodiments of the methods disclosed herein, the subject exhibits acquired resistance or intrinsic resistance to tazemetostat. In any of the preceding embodiments of the methods disclosed herein, the subject is a child or an adult.

Additionally or alternatively, in some embodiments of the methods disclosed herein, the CDK4/6 inhibitor, the AURK inhibitor, or the ATR inhibitor is sequentially, simultaneously, or separately administered with tazemetostat. In some embodiments, the CDK4/6 inhibitor, the AURK inhibitor, the ATR inhibitor or tazemetostat is administered orally, intravenously, intramuscularly, intraperitoneally, or subcutaneously.

In one aspect, the present disclosure provides a method for selecting a sarcoma patient for treatment with tazemetostat comprising detecting mRNA or polypeptide expression and/or activity levels of KLF4 in a biological sample obtained from the sarcoma patient that are elevated compared to a control sample obtained from a healthy subject or a predetermined threshold; and administering to the sarcoma patient an effective amount of tazemetostat. In one aspect, the present disclosure provides a method for selecting a sarcoma patient for treatment with tazemetostat and an additional therapeutic agent comprising detecting mRNA or polypeptide expression and/or activity levels of one or more of PRICKLE1, PLK1, and CELSR2 in a biological sample obtained from the sarcoma patient that are elevated compared to a control sample obtained from a healthy subject or a predetermined threshold; and administering to the sarcoma patient an effective amount of tazemetostat and the additional therapeutic agent, wherein the additional therapeutic agent is an ATR inhibitor, an AURK inhibitor or a CDK4/6 inhibitor. In another aspect, the present disclosure provides a method for selecting a sarcoma patient for treatment with tazemetostat and an additional therapeutic agent comprising detecting mRNA or polypeptide expression and/or activity levels of KLF4 in a biological sample obtained from the sarcoma patient that are reduced compared to a control sample obtained from a healthy subject or a predetermined threshold; and administering to the sarcoma patient an effective amount of tazemetostat and the additional therapeutic agent, wherein the additional therapeutic agent is an ATR inhibitor, an AURK inhibitor or a CDK4/6 inhibitor. In some embodiments, mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). Additionally or alternatively, in some embodiments, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

Also disclosed herein are kits comprising (a) tazemetostat, (b) at least one of a CDK4/CDK6 inhibitor, an AURK inhibitor, and AT R inhibitor, and (c) instructions for treating sarcomas.

### BRIEF DESCRIPTION OF THE DRAWINGS

FIGS. 1A-1E: Patient tumor sequencing reveals diverse tazemetostat resistance mutations. FIG. 1A: Abridged oncoprint of selected genes from MSK-IMPACT sequencing on patient tumor samples. Top panel: Tumor samples prior to TAZ treatment. Bottom panel: Matched samples pre- and post-TAZ or pre- and post-acquisition of resistance. FIG. 1B: Pre- and post-treatment CT imaging of the indicated patient tumors which acquired EZH2 and RB1 mutations. FIG. 1C: Atomic molecular model of the chimeric Homo sapiens/Anolis carolinensis EZH2 bound to pyridone-based EZH2 inhibitor I (blue), PDB: 5IJ7. Y666 is highlighted in red. FIG. 1D: Top panel: Doxycycline-inducible EZH2 is expressed in single-cell G401 clones at near-physiological levels after 3 days treatment with doxycycline at 1 μg/mL. Numbers indicate clone ID. Bottom panel: Cell viability measured by CellTiter-Glo after 14 days of treatment with the indicated drug at 10 μM or equivalent volume of DMSO. n=5 biological replicates per condition. *p=3.5E-3, **p=5.2E-3, ***p=3.8E-5, ****p=1.1E-5 by two-sided Student's t-test. FIG. 1E: Top panel: RB1 knockout in two G401 clones (E1 and F2). Bottom panel: Cell viability measured by CellTiter-Glo after treatment with 10 μM tazemetostat or DMSO for 14 days. n=5 biological replicates per condition. *p=2.8E-5, **p=9.0E-5 by two-sided Student's t-test.

FIGS. 2A-2H: RB1 loss allows escape from cell cycle arrest despite effective EZH2 inhibition. FIGS. 2A-2C: Volcano plots of RNA-seq data from TAZ-treated G401 RB1^{WT} (FIG. 2A), RB1^{del} E1 (FIG. 2B), and RB1^{del} F2 (FIG. 2C). Plots show gene expression changes of cells treated with 10 μM TAZ versus equivalent volume of DMSO for 11 days. n=3 biological replicates per condition. Dots in red show genes with expression changes of log₂(fold change) >±1 and p-value <0.01. FIGS. 2D-2F: GSEA plots showing the PRC2_EZH2_UP.UP gene set for G401 RB1^{WT} (FIG. 2D), RB1^{del} E1 (FIG. 2E), and RB1^{del} F2 (FIG. 2F) cells treated in FIGS. 2A-2C. FIG. 2G: Cell cycle analysis of the indicated G401 clone. Plot shows triplicate measurements of cells treated with 1 μM TAZ versus equivalent volume of DMSO for 11 days. Y-axis shows the percent difference of the TAZ-treated cells in each cell cycle phase compared to DMSO-treated cells. FIG. 2H: Western blot of the indicated G401 cell clones treated with 10 μM TAZ versus equivalent volume of DMSO for 11 days. Actin serves as loading control.

FIGS. 3A-3H: An intact RB1/E2F axis is a key requirement for response to TAZ: FIG. 3A: Dose-response curves of a panel of MRT and ES cell lines treated with TAZ for 13 days. Curves in red correspond to TAZ-responsive cell lines, grey to TAZ-resistant cell lines. FIG. 3B: Abridged oncoprint of MRT and ES cell lines. Genes included here are BAF subunits and CDK4/6/RB1/E2F axis genes. FIGS. 3C-3D: Volcano plots of RNA-seq data comparing DMSO-treated G401 RB1^{del} E1 (FIG. 3C) and F2 (FIG. 3D) with RB1^{WT} cells. FIG. 3E: Western blot for PRICKLE1 in untreated G401 cells. FIGS. 3F-3G: Read counts for PRICKLE1 (FIG. 3F) and KLF4 (FIG. 3G) showing counts normalized by DESeq2 for patient tumor samples collected prior to TAZ treatment. FIG. 3H: Read counts for AURKB for patient tumor samples collected after TAZ treatment. *p=0.013, **p=0.03.

FIGS. 4A-4F: Cell cycle bypass combination strategy using AURKB inhibition overcomes TAZ resistance and improves response: FIG. 4A: G401 cells treated with barasertib for 6 days (FIG. 4A). FIG. 4B: Panel of MRT and ES cell lines ordered left to right by decreasing response to TAZ monotherapy. Cells were treated with the indicated monotherapy or combination for 11 days. Drug concentrations used were: TAZ: 200 nM, barasertib: 8 nM. FIG. 4C: Tumor growth curves showing volumes calculated from caliper measurements for 5 mouse PDXs treated with the indicated drug regimen. n=20 mice for vehicle and barasertib-treated groups, n=21 for TAZ and TAZ+barasertib-treated groups. Vardi U-test p=4.0E-4 and 2.0E-4 for combination vs. barasertib or TAZ, respectively. FIG. 4D: Kaplan-Meier curves showing tumor-free survival (defined as tumor volume ≤1,000 mm³) for the PDXs in panel C. Mean survival is 65 days (95% CI: 51-78 days) for barasertib, 67 days (95% CI: 53-81 days) for TAZ, 98 days (95% CI: 84-112 days) for the combination. Log-rank test p=3.3E-3 and 5.8E-3 for combination vs. barasertib or TAZ, respectively. FIG. 4E: Tumor growth curves for the subset of mouse tumors in panel C from the SOMWR_EPIS_X00013aS1 PDX model. n=3 mice per treatment group. Vardi U-test p=0.10 and 9.4E-2 for combination vs. barasertib or TAZ, respectively. FIG. 4F: Tumors from panel E harvested on Day 135 of treatment.

FIGS. 5A-5H: Synthetic lethal combination strategy using ATR inhibition overcomes TAZ resistance and improves response: FIG. 5A: G401 cells treated with elimusertib for 4 days. FIG. 5B: Panel of MRT and ES cell lines ordered left to right by decreasing response to TAZ monotherapy. Cells were treated with the indicated monotherapy or combination for 11 days. Drug concentrations used were: TAZ: 200 nM, elimusertib: 8 nM. We selected an elimusertib dose below its monotherapy IC₅₀ (for G401 cells) in order to visualize any additive effects upon combination with TAZ. FIG. 5C: Representative images of G401 cells treated with the indicated treatment for 7 days. FIG. 5D: Quantification of γH2AX fluorescence relative to DAPI fluorescence. *p=0.042, **p=5.1E-3, ***p=1.7E-8, ****p=6.5E-4 by two-sided Student's t-test. n=548 nuclei for DMSO, 432 for elimusertib, 696 for 50 nM TAZ, 401 for 50 nM TAZ+elimusertib, 373 for 100 nM TAZ, 63 for 100 nM TAZ+elimusertib. FIG. 5E: Tumor growth curves for 5 mouse PDXs treated with the indicated drug regimen. n=20 mice for vehicle and elimusertib-treated groups, n=21 for TAZ and TAZ+elimusertib-treated groups. Vardi U-test p=3.2E-2 and 0.23 for combination vs. elimusertib or TAZ, respectively. FIG. 5F: Kaplan-Meier curves showing tumor-free survival (defined as tumor volume ≤1,000 mm³) for the PDXs in panel C. Mean survival is 51 days (95% CI: 42-60 days) for elimusertib, 67 days (95% CI: 53-81 days) for TAZ to 99 days (95% CI: 74-123 days) for the combination. Log-rank test p=5.8E-4 and 3.9E-2 for combination versus elimusertib or TAZ, respectively. FIG. 5G: Tumor growth curves for the HYMAD_EPIS_X0004aS1 PDX model treated with the indicated drug regimen. Vardi U-test p=2.0E-4 for combination versus elimusertib or TAZ. n=14 mice per treatment group. FIG. 5H: Kaplan-Meier curves showing tumor-free survival (defined as tumor volume ≤1,000 mm³) for the PDXs in panel E. Log-rank test p=6.2E-3 and 6.3E-5 for combination versus elimusertib or TAZ, respectively.

FIG. 6: Mechanistic schematic for the response of BAF-deficient tumors to effective EZH2 therapy: Step 1: TAZ inhibits histone methylation activity of PRC2. Step 2: Activating chromatin-bound complexes, such as ncBAF bind to tumor suppressor loci. Step 3: Tumor suppressor loci are activated by their transcription factors and their coactivators. Step 4: Tumor suppressors inhibit cell cycle progression through their downstream effectors, such as RB1/E2F.

FIGS. 7A-7E: Validation of tumor resistance mutations of EZH2: FIGS. 7A-7B: Integrated Genome Viewer (IGV) tracks of RNA-seq data for post-treatment tumor samples from patients 3 and 15. Purple boxes indicate the mutation sites. FIG. 7A: Aligned reads and coverage plot of exon 16 of EZH2, showing expression of mRNA with the T (red)→A (green) mutant allele. Figure discloses SEQ ID NOS 16-25, 21, 24, 26-27, 24, 28, 24, 26, 21, 29, 21, 21, 30, 26, 31, 21, 32, 26, 31, 33, 34, 16, 34, 34, 34, and 34-36 respectively, in order of appearance. FIG. 7B: Aligned reads and coverage plot of exon 3 of RB1, showing expression of mRNA with frame shift deletion at 1124. Figure discloses SEQ ID NOS 37, 37, 38, 38, 37, 37, 37, 39, 40, 37, 37, 41, 41, 42, 37, 37, 37, 37, 37, 37, 37, 38, 38, 43, 44 and 44 respectively, in order of appearance. FIG. 7C: Phase-contrast microscopy of G401 single-cell clones expressing the indicated form of EZH2. Cells were treated with 10 μM tazemetostat or DMSO for 9 days and imaged with an Evos FL Auto 2 imager at 10× magnification. Arrow indicates a refractile, mitotic cell. Arrowhead indicates a post-treatment, morphologically altered cell. FIG. 7D: Cell viability measured by CellTiter-Glo after 14 days of treatment with 10 μM tazemetostat or equivalent volume of DMSO. Data is the same as in FIG. 1D with the addition of EZH2^{CatMut} and EZH2^{QuadMut} clones. n=5 replicates per condition. FIG. 7E: Cell viability after treatment with 10 μM valemetostat or DMSO for 14 days. n=5 replicates per condition.

FIGS. 8A-8F: RB1^{del} cells show morphological and transcriptional responses to TAZ: FIG. 8A: Phase-contrast microscopy of G401 cells with or without RB1 expression. Cells were treated with 10 μM tazemetostat or DMSO for 9 days and imaged with an Evos FL Auto 2 imager at 10× magnification. Arrow indicates a refractile, mitotic cell. Arrowhead indicates a post-treatment, morphologically altered cell. FIG. 8B: Western blot of the indicated G401 cell clones treated with 10 μM TAZ vs. equivalent volume of DMSO for 11 days. Bulk H3K27me3 levels are reduced in all three clones despite persistent EZH2 expression in RB1^{del} clones. FIG. 8C: Comparison of all Hallmark gene sets upregulated in G401 cells upon TAZ treatment with significance at FDR<25%. FIGS. 8D-8E: GSEA plots showing the Hallmark_E2F_Targets gene set comparing TAZ-treated RB1^{del} G401 cells with RB1^{WT} cells. FIG. 8F: GSEA plots showing the Hallmark_Epithelial_Mesenchymal_Transition gene set for the indicated TAZ-treated G401 cells compared to DMSO.

FIGS. 9A-9C: Characterization of MRT and ES cell lines: FIG. 9A: Confirmed loss of SMARCB1 expression in all MRT and ES cell lines used. FIG. 9B: Plot of area under the curve (AUC) of the dose-response curves from FIG. 3A plotted against doubling time. AUC was integrated from 40 nM to 50 μM (FIG. 9C) IGV track of reads from MSK-IMPACT sequencing of KP-MRT-RY cells, focusing on the RB1 gene, shows reads spanning segments across the full gene and indicates the presence of at least one allele.

FIGS. 10A-10D: TAZ-resistant patient tumors show upregulation of cell cycle genes: Top 30 GO terms, sorted by p-value, enriched in pre-treatment (FIG. 10A) and post-treatment (FIG. 10B) patient tumor specimens that did not respond to TAZ, compared to those that did. (FIGS. 10C-10D) DESeq2-normalized read counts of genes from the indicated GO terms comparing pre-treatment TAZ-responding tumors to pre-treatment non-responding tumors.

FIGS. 11A-11C: Transcriptomic analysis of patient tumors nominates putative biomarkers of TAZ sensitivity and resistance: FIG. 11A: Venn diagrams of genes up- or down-regulated by RB1 knockout in the indicated clone. Same data as in FIG. 2. FIGS. 11B-11C: Heatmaps showing the top 60 upregulated (FIG. 11B) and downregulated (FIG. 11C) genes in patient tumors collected prior to TAZ-treatment. Each column indicates a separate tumor. Heatmaps are sorted by t-statistic calculated using a two-tailed Student's t-test, p<0.05. Red boxes indicate planar cell polarity genes CELSR2, PLK1, and PRICKLE1 and CDKN1A regulator KLF4.

FIGS. 12A-12F: Downstream cell cycle inhibitors overcome resistance to TAZ: G401 cells treated with seleciclib (FIG. 12A), alisertib (FIG. 12B), abemaciclib (FIG. 12C), or palbociclib (FIG. 12D) 9 days. FIGS. 12E-12F: Synergy plots for combination treatment with TAZ and elimusertib for (FIG. 12E) G401 and (FIG. 12F) ES1 cells. Cells were treated at the indicated doses for 9 days, and analyzed for synergy using the Zero Interaction Potency (ZIP) model.

FIGS. 13A-13B: TAZ induces DNA damage in G401 cells: FIG. 13A: Quantification of γH2AX fluorescence relative to DAPI fluorescence using CellProfiler. n=53, 548, and 432 nuclei for negative control, DMSO, and elimusertib, respectively. FIG. 13B: Representative uncropped images of G401 cells treated as indicated (same images as in FIG. 5C). 3 field are shown per condition. Scale bars are 50 μm.

FIGS. 14A-14C: Response of individual PDX models to combination therapy: FIG. 14A: Tumor growth curves for the subset of mouse tumors in FIG. 4C from the HYMAD_EPIS_X0003aS1 PDX model. n=2 mice for vehicle and barasertib-treated groups, n=3 mice for TAZ and TAZ+barasertib-treated groups. FIG. 14B: Image of representative tumors extracted from mice in FIG. 5G and on Day 52 of treatment (FIG. 14C) their corresponding weights. *p=6.7E-3 by two-sided Student's t-test.

FIGS. 15A-15C: CHK1 inhibition does not induce replication stress or synergize with TAZ. FIG. 15A: Dose-response curves of G401 cells treated with the CHK1 inhibitor SRA737 for 9 days. FIG. 15B: Western blot assaying replication stress as measured by RPA phosphorylation at S4/8 and T21. Camptothecin treatment (1.5 μM) for 2 h was used as a positive control for replication stress. Autophosphorylation of CHK1 at S296 was used to confirm CHK1 inhibition. Cells were pre-treated with 10 μM TAZ or DMSO for 9 days. Cells were then split and additionally treated with SRA737 (3 μM) or equivalent volume of DMSO for 2 days. FIG. 15C: Cells treated with 10 μM TAZ or DMSO for 11 days do not express MYCN protein. MYCN-amplified neuroblastoma cell line IMR5 was used as a positive control for MYCN expression.

FIG. 16: TAZ may remodel BAF and PRC2 composition by transcriptional regulation of their subunits: DESeq2-normalized read counts for all BAF and PRC2 subunits showing significantly altered gene expression between TAZ and DMSO-treated cells. Same data as in FIG. 2.

FIG. 17A shows the chemical structure of ATR inhibitor elimusertib. FIG. 17B shows the activity of ATR inhibitor elimusertib in combination with tazemetostat at different doses. Synergy Scores calculated using Zero-Interaction Potency (ZIP) model, implemented in SynergyFinder package (Tang et al, Front Pharmacol, 6, 181 (2015)). Without wishing to be bound by theory, it is believed that tazemetostat (EPZ-6438) sensitizes cells to ATR inhibition due to EZH2-mediated upregulation of PGBD5, which creates dsDNA breaks.

FIG. 18A shows the chemical structure of CDK4/6 inhibitor palbociclib. FIG. 18B shows the activity of CDK4/6 inhibitor palbociclib in combination with tazemetostat at different doses. Synergy Scores calculated using Zero-Interaction Potency (ZIP) model, implemented in SynergyFinder package (Tang et al, Front Pharmacol, 6, 181 (2015)). Without wishing to be bound by theory, it is believed that CDK4/6 inhibition shows synergy with tazemetostat (EPZ-6438) through a “double-hit” on the CDK4/6-RB-E2F pathway.

FIG. 19A shows the chemical structure of CDK4/6 inhibitor abemaciclib. FIG. 19B shows the activity of CDK4/6 inhibitor abemaciclib in combination with tazemetostat at different doses. Synergy Scores calculated using Zero-Interaction Potency (ZIP) model, implemented in SynergyFinder package (Tang et al, Front Pharmacol, 6, 181 (2015)). Without wishing to be bound by theory, it is believed that CDK4/6 inhibition shows synergy with tazemetostat (EPZ-6438) through a “double-hit” on the CDK4/6-RB-E2F pathway.

FIG. 20A shows the chemical structure of AURKB inhibitor barasertib. FIG. 20B shows the activity of AURKB inhibitor barasertib in combination with tazemetostat at different doses. Synergy Scores calculated using Zero-Interaction Potency (ZIP) model, implemented in SynergyFinder package (Tang et al, Front Pharmacol, 6, 181 (2015)). Without wishing to be bound by theory, it is believed that tazemetostat (EPZ-6438) sensitizes cells to AURKB inhibition through downregulation of AURKB, even as cells continue to transit through the cell cycle.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, immunology, microbiology and recombinant DNA are used. See, e.g., Sambrook and Russell eds. (2001)Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, intrathecally, parenterally (intravenously, intramuscularly, intraperitoneally, or subcutaneously), rectally, intrathecally, intraocularly, intradermally, transmucosally, iontophoretically, or topically. Administration includes self-administration and the administration by another.

As used herein, the terms “ATR” or “ataxia telangiectasia and Rad3-related protein” refer to a serine/threonine-specific protein kinase that is involved in sensing DNA damage and activating the DNA damage checkpoint, leading to cell cycle arrest in eukaryotes.

The term “ATR inhibitor” refers to a compound that is capable of interacting with, and inhibiting the enzymatic activity of ATR. As used herein, inhibiting ATR enzymatic activity means reducing the ability of ATR to phosphorylate a substrate peptide or protein. In some embodiments, the ATR inhibitor reduces ATR enzymatic activity by at least about 50%, at least about 75%, at least about 90%, at least about 95%, or at least about 99%. In various embodiments, the concentration of ATR inhibitor required to reduce ATR enzymatic activity is less than about 1 M, less than about 500 nM, less than about 100 nM, or less than about 50 nM. In some embodiments, the ATR inhibitor is selective, e.g., the ATR inhibitor reduces the ability of ATR to phosphorylate a substrate peptide or protein at a concentration that is lower than the concentration of the inhibitor that is required to produce another, unrelated biological effect, e.g., reduction of the enzymatic activity of a different kinase.

As used herein, the terms “AURK” or “Aurora kinases” refer to a family of highly conserved serine/threonine kinases that are important for faithful transition through mitosis. In humans, the Aurora kinase family consists of three members; Aurora-A (AURKA), Aurora-B (AURKB), and Aurora-C (AURKC), which each share a conserved C-terminal catalytic domain but differ in their sub-cellular localization, substrate specificity, and function during mitosis. In addition, Aurora-A and Aurora-B have been found to be overexpressed in a wide variety of human tumors.

The term “AURK inhibitor” refers to a compound that is capable of interacting with, and inhibiting the enzymatic activity of an Aurora Kinase (AURK). As used herein, inhibiting AURK enzymatic activity means reducing the ability of AURK to phosphorylate a substrate peptide or protein. In some embodiments, the AURK inhibitor reduces AURK enzymatic activity by at least about 50%, at least about 75%, at least about 90%, at least about 95%, or at least about 99%. In various embodiments, the concentration of AURK inhibitor required to reduce AURK enzymatic activity is less than about 1 M, less than about 500 nM, less than about 100 nM, or less than about 50 nM. In some embodiments, the AURK inhibitor is selective, e.g., the AURK inhibitor reduces the ability of AURK to phosphorylate a substrate peptide or protein at a concentration that is lower than the concentration of the inhibitor that is required to produce another, unrelated biological effect, e.g., reduction of the enzymatic activity of a different kinase.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, a “sample” or “biological sample” refers to a body fluid or a tissue sample isolated from a subject. In some cases, a biological sample may consist of or comprise whole blood, platelets, red blood cells, white blood cells, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, endothelial cells, synovial fluid, lymphatic fluid, ascites fluid, interstitial or extracellular fluid and the like. The term “sample” may also encompass the fluid in spaces between cells, including gingival crevicular fluid, bone marrow, cerebrospinal fluid (CSF), saliva, mucus, sputum, semen, sweat, urine, or any other bodily fluids. Samples can be obtained from a subject by any means including, but not limited to, venipuncture, excretion, ejaculation, massage, biopsy, needle aspirate, lavage, scraping, surgical incision, or intervention or other means known in the art. A blood sample can be whole blood or any fraction thereof, including blood cells (red blood cells, white blood cells or leukocytes, and platelets), serum and plasma.

As used herein, the term “separate” therapeutic use refers to an administration of at least two active ingredients at the same time or at substantially the same time by different routes.

As used herein, the term “sequential” therapeutic use refers to administration of at least two active ingredients at different times. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject”, “patient”, or “individual” can be an individual organism, a vertebrate, a mammal, or a human. In some embodiments, the subject, patient or individual is a human.

As used herein, a “synergistic therapeutic effect” refers to a greater-than-additive therapeutic effect which is produced by a combination of at least two agents, and which exceeds that which would otherwise result from the individual administration of the agents. For example, lower doses of one or more agents may be used in treating a disease or disorder, resulting in increased therapeutic efficacy and decreased side-effects.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Sarcomas

Sarcomas are refractory tumors that tend to occur in children and young adults. In particular, rhabdoid tumors and epithelioid sarcomas that cannot be cured with surgery are resistant to all known forms of therapy and almost uniformly lethal. Recent studies have revealed essential functions of dysregulated gene expression in sarcoma development, and mutagenic activity of the PGBD5 DNA transposase in promoting oncogenic mutations in rhabdoid tumors, including deletions of SMARCB1, a key component of the BAF chromatin remodeling complex, which is dysregulated to cause this disease.

### Tazemetostat

EZH2 is the catalytic subunit of the polycomb repressive complex 2 (PRC2) that functions as a histone methyltransferase. PRC2 is part of a multiprotein complex that regulates cell development through chromatin compaction and gene repression. As an enzyme within this complex, EZH2 works through PRC2-dependent trimethylation of histone 3 lysine 27 (H3K27). Methylation of H3K27 leads to gene repression and is a major epigenetic phenomenon during tissue development and stem cell determination. EZH2 works as a master regulator of cell cycle progression, autophagy, apoptosis, and promotes DNA damage repair and inhibits cellular senescence

Tazemetostat is a first-in-class targeted epigenetic regulator that specifically inhibits EZH2. This FDA-approved oral treatment received accelerated approval for patients with hematologic and solid malignancies. Tazemetostat was first approved for patients 16 years and older with metastatic or locally advanced epithelioid sarcoma not eligible for complete resection based on the results of an international open-label phase II basket trial. Another open-label multicenter phase II trial led to the approval for patients with relapsed or refractory follicular lymphoma with EZH2 mutation who have received at least two prior systemic therapies or patients who have no satisfactory alternative treatment options.

### ATR Inhibitors

ATR (“ATM and Rad3 related”) is a key mediator of cellular responses to a DNA damage structure that is characterized by tracts of single stranded DNA coated by replication protein A (RPA). RPA coated single stranded DNA commonly arises as a result of replication stress, which occurs when a cell attempts to replicate DNA through unresolved DNA damage lesions. Failure to repair replication stress can lead to lethal double strand breaks or deleterious DNA mutation. ATR along with its substrates coordinates multiple cell functions in response to replication stress including cell cycle control, DNA replication and DNA damage repair.

ATR inhibitors can be selective or non-selective, small molecules or biomolecules. In some embodiments, the ATR inhibitor is selected from the group consisting of elimusertib (BAY1895344), Schisandrin B, NU6027, dactolisib (NVP-BEZ235), EPT-46464, Torin 2, VE-821, AZ20, M4344 (VX-803), berzosertib (M6620 (VX-970)), and ceralasertib (AZD6738).

### AURK Inhibitors

The Aurora kinase family comprises of cell cycle-regulated serine/threonine kinases important for mitosis. Their activity and protein expression are cell cycle regulated, peaking during mitosis to orchestrate important mitotic processes including centrosome maturation, chromosome alignment, chromosome segregation, and cytokinesis. In humans, the Aurora kinase family consists of three members; Aurora-A, Aurora-B, and Aurora-C, which each share a conserved C-terminal catalytic domain but differ in their sub-cellular localization, substrate specificity, and function during mitosis. In addition, Aurora-A and Aurora-B have been found to be overexpressed in a wide variety of human tumors.

The AURK inhibitor may be a selective inhibitor of AURKB or a pan AURK inhibitor. In some embodiments, the AURK inhibitor is selected from the group consisting of tozasertib, SP-96, AT9283, 549076, danusertib (PHA-739358), PF-03814735, chiauranib, AMG900, Alisertib, cenisertib, SNS-314, barasertib, hesperadin, AZD1152, GSK1070916, CYC116, BI 811283, AZD2811, PHA680632, reversine, CCT129202, CCT137690, SNS-314, quercetin, and VX-680.

### CDK4/CDK6 Inhibitors

CDK4 and CDK6 are cyclin-dependent kinases that control the transition between the G1 and S phases of the cell cycle. The S phase is the period during which the cell synthesizes new DNA and prepares itself to divide during mitosis. CDK4/6 activity is typically deregulated and overactive in cancer cells. Some cancers exhibit amplification or overexpression of the genes encoding cyclins or the CDKs themselves.

A major target of CDK4 and CDK6 during cell-cycle progression is the retinoblastoma protein (RB). When RB is phosphorylated, its tumor-suppressive properties are inactivated. Selective CDK4/6 inhibitors deactivate CDK4 and CDK6 and dephosphorylate RB, resulting in cell-cycle arrest. In some cases, the arrested cells enter a state of senescence. Examples of CDK4/6 inhibitors include palbociclib, ribociclib, and abemaciclib.

Formulations Including Tazemetostat, CDK4/6 Inhibitors, ATR Inhibitors and/or AURK Inhibitors of the Present Technology

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### Methods of Treatment of the Present Technology

In one aspect, the present disclosure provides a method for selecting a sarcoma patient for treatment with tazemetostat comprising detecting mRNA or polypeptide expression and/or activity levels of KLF4 in a biological sample obtained from the sarcoma patient that are elevated compared to a control sample obtained from a healthy subject or a predetermined threshold; and administering to the sarcoma patient an effective amount of tazemetostat. In one aspect, the present disclosure provides a method for selecting a sarcoma patient for treatment with tazemetostat and an additional therapeutic agent comprising detecting mRNA or polypeptide expression and/or activity levels of one or more of PRICKLE1, PLK1, and CELSR2 in a biological sample obtained from the sarcoma patient that are elevated compared to a control sample obtained from a healthy subject or a predetermined threshold; and administering to the sarcoma patient an effective amount of tazemetostat and the additional therapeutic agent, wherein the additional therapeutic agent is an ATR inhibitor, an AURK inhibitor or a CDK4/6 inhibitor. In another aspect, the present disclosure provides a method for selecting a sarcoma patient for treatment with tazemetostat and an additional therapeutic agent comprising detecting mRNA or polypeptide expression and/or activity levels of KLF4 in a biological sample obtained from the sarcoma patient that are reduced compared to a control sample obtained from a healthy subject or a predetermined threshold; and administering to the sarcoma patient an effective amount of tazemetostat and the additional therapeutic agent, wherein the additional therapeutic agent is an ATR inhibitor, an AURK inhibitor or a CDK4/6 inhibitor. In some embodiments, mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). Additionally or alternatively, in some embodiments, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

In one aspect, the present disclosure provides a method for treating sarcoma in a subject in need thereof comprising administering to the subject an effective amount of tazemetostat and an effective amount of a CDK4/6 inhibitor. In some embodiments, the CDK4/6 inhibitor is selected from the group consisting of palbociclib, ribociclib, and abemaciclib.

In one aspect, the present disclosure provides a method for treating sarcoma in a subject in need thereof comprising administering to the subject an effective amount of tazemetostat and an effective amount of an AURK inhibitor. The AURK inhibitor may be a selective inhibitor of AURKB or a pan AURK inhibitor. Examples of AURK inhibitors include, but are not limited to tozasertib, SP-96, AT9283, danusertib (PHA-739358), AMG900, cenisertib, SNS-314, barasertib, hesperadin, AZD1152, GSK1070916, CYC116, BI 811283, AZD2811, PHA680632, reversine, CCT129202, CCT137690, S49076, PF-03814735, chiauranib, Alisertib, quercetin, and VX-680.

In another aspect, the present disclosure provides a method for treating sarcoma in a subject in need thereof comprising administering to the subject an effective amount of tazemetostat and an effective amount of an ATR inhibitor. Examples of ATR inhibitors include, but are not limited to elimusertib (BAY1895344), Schisandrin B, NU6027, dactolisib (NVP-BEZ235), EPT-46464, Torin 2, VE-821, AZ20, M4344 (VX-803), berzosertib (M6620 (VX-970)), and ceralasertib (AZD6738).

Additionally or alternatively, in some embodiments of the methods disclosed herein, the subject is non-responsive to at least one prior line of cancer therapy such as chemotherapy or immunotherapy. In certain embodiments, the subject is human.

In any and all embodiments of the methods disclosed herein, the sarcoma is a rhabdoid sarcoma or an epithelioid sarcoma. In some embodiments, the sarcoma comprises a deficiency in a BAF chromatin remodeling complex component or SMARCB1. Additionally or alternatively, in some embodiments, the sarcoma is derived from soft tissue trunk, mediastinal, pelvis, stomach, lymph node, soft tissue, kidney, retroperitoneal soft tissue, posterior chest wall, perineal mass, thigh, arm, breast, groin, ovary, abdomen, or distal humerus.

Additionally or alternatively, in some embodiments, the subject comprises an acquired mutation in EZH2, CDKN2A B, CDKN1A, ANKRD11 or RB1. In any and all embodiments of the methods disclosed herein, the subject exhibits acquired resistance or intrinsic resistance to tazemetostat. In any of the preceding embodiments of the methods disclosed herein, the subject is a child or an adult.

Additionally or alternatively, in some embodiments of the methods disclosed herein, the CDK4/6 inhibitor, the AURK inhibitor, or the ATR inhibitor is sequentially, simultaneously, or separately administered with tazemetostat. In some embodiments, the CDK4/6 inhibitor, the AURK inhibitor, the ATR inhibitor or tazemetostat is administered orally, intravenously, intramuscularly, intraperitoneally, or subcutaneously.

Additionally or alternatively, in some embodiments of the combination therapy methods disclosed herein, the time to response and/or duration of response is improved relative to that observed with tazemetostat monotherapy or monotherapy with an AURK inhibitor, an ATR inhibitor, or CDK4/6 inhibitor.

Additionally or alternatively, in some embodiments of the methods disclosed herein, the tazemetostat and the CDK4/6 inhibitor are administered sequentially, simultaneously, or separately. The tazemetostat and/or the CDK4/6 inhibitor may be administered orally, parenterally, by inhalation spray, intranasally, buccally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intravenously, or subcutaneously. Formulations including tazemetostat and/or CDK4/6 inhibitor disclosed herein may be designed to be short-acting, fast-releasing, or long-acting. In other embodiments, compounds can be administered in a local rather than systemic means, such as administration (e.g., by injection) at a tumor site.

Additionally or alternatively, in some embodiments of the methods disclosed herein, the tazemetostat and the ATR inhibitor are administered sequentially, simultaneously, or separately. The tazemetostat and/or the ATR inhibitor may be administered orally, parenterally, by inhalation spray, intranasally, buccally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intravenously, or subcutaneously. Formulations including tazemetostat and/or ATR inhibitor disclosed herein may be designed to be short-acting, fast-releasing, or long-acting. In other embodiments, compounds can be administered in a local rather than systemic means, such as administration (e.g., by injection) at a tumor site.

Additionally or alternatively, in some embodiments of the methods disclosed herein, the tazemetostat and the AURK inhibitor are administered sequentially, simultaneously, or separately. The tazemetostat and/or the AURK inhibitor may be administered orally, parenterally, by inhalation spray, intranasally, buccally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intravenously, or subcutaneously. Formulations including tazemetostat and/or AURK inhibitor disclosed herein may be designed to be short-acting, fast-releasing, or long-acting. In other embodiments, compounds can be administered in a local rather than systemic means, such as administration (e.g., by injection) at a tumor site.

Additionally or alternatively, in some embodiments of the methods disclosed herein, tazemetostat can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), simultaneously with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an AURK inhibitor, an ATR inhibitor, or a CDK4/6 inhibitor to a patient with sarcoma.

In some embodiments, tazemetostat and at least one of an AURK inhibitor, an ATR inhibitor, or a CDK4/6 inhibitor are administered to a patient, for example, a mammal, such as a human, in a sequence and within a time interval such that the inhibitor that is administered first acts together with the inhibitor that is administered second to provide greater benefit than if each inhibitor were administered alone. For example, tazemetostat and at least one of an AURK inhibitor, an ATR inhibitor, or a CDK4/6 inhibitor can be administered at the same time or sequentially in any order at different points in time; however, if not administered at the same time, tazemetostat and at least one of an AURK inhibitor, an ATR inhibitor, or a CDK4/6 inhibitor are administered sufficiently close in time so as to provide the desired therapeutic or prophylactic effect of the combination of the at least two inhibitors. In one embodiment, tazemetostat and at least one of an AURK inhibitor, an ATR inhibitor, or a CDK4/6 inhibitor exert their effects at times which overlap. In some embodiments, tazemetostat and at least one of an AURK inhibitor, an ATR inhibitor, or a CDK4/6 inhibitor are each administered as separate dosage forms, in any appropriate form and by any suitable route. In other embodiments, tazemetostat and at least one of an AURK inhibitor, an ATR inhibitor, or a CDK4/6 inhibitor are administered simultaneously in a single dosage form.

It will be appreciated that the frequency with which any of these therapeutic agents can be administered can be once or more than once over a period of about 2 days, about 3 days, about 4 days, about 5 days, about 6 days, about 7 days, about 8 days, about 9 days, about 10 days, about 11 days, about 12 days, about 13 days, about 14 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every year, about every 2 years, about every 3 years, about every 4 years, or about every 5 years.

For example, tazemetostat, an AURK inhibitor, an ATR inhibitor or a CDK4/6 inhibitor may be administered daily, weekly, biweekly, or monthly for a particular period of time. Tazemetostat, an AURK inhibitor, an ATR inhibitor or a CDK4/6 inhibitor may be dosed daily over a 14 day time period, or twice daily over a seven day time period. Tazemetostat, an AURK inhibitor, an ATR inhibitor or a CDK4/6 inhibitor may be administered daily for 7 days.

Alternatively, tazemetostat, an AURK inhibitor, an ATR inhibitor or a CDK4/6 inhibitor may be administered daily, weekly, biweekly, or monthly for a particular period of time followed by a particular period of non-treatment. In some embodiments, tazemetostat, an AURK inhibitor, an ATR inhibitor or a CDK4/6 inhibitor can be administered daily for 14 days followed by seven days of non-treatment, and repeated for two more cycles of daily administration for 14 days followed by seven days of non-treatment. In some embodiments, tazemetostat, an AURK inhibitor, an ATR inhibitor or a CDK4/6 inhibitor can be administered twice daily for seven days followed by 14 days of non-treatment, which may be repeated for one or two more cycles of twice daily administration for seven days followed by 14 days of non-treatment.

In some embodiments, tazemetostat, the AURK inhibitor, the ATR inhibitor or the CDK4/6 inhibitor is administered daily over a period of 14 days. In another embodiment, tazemetostat, the AURK inhibitor, the ATR inhibitor or the CDK4/6 inhibitor is administered daily over a period of 12 days, or 11 days, or 10 days, or nine days, or eight days. In another embodiment, tazemetostat, the AURK inhibitor, the ATR inhibitor or the CDK4/6 inhibitor is administered daily over a period of seven days. In another embodiment, tazemetostat, the AURK inhibitor, the ATR inhibitor or the CDK4/6 inhibitor is administered daily over a period of six days, or five days, or four days, or three days.

In some embodiments, individual doses of tazemetostat and the CDK4/6 inhibitor, AURK inhibitor, or ATR inhibitor are administered within a time interval such that the two inhibitors can work together (e.g., within 1 hour, 2 hours, 4 hours, 6 hours, 12 hours, 24 hours, 48 hours, 72 hours, 96 hours, 5 days, 6 days, 1 week, or 2 weeks). In some embodiments, the treatment period during which the therapeutic agents are administered is then followed by a non-treatment period of a particular time duration, during which the therapeutic agents are not administered to the patient. This non-treatment period can then be followed by a series of subsequent treatment and non-treatment periods of the same or different frequencies for the same or different lengths of time. In some embodiments, the treatment and non-treatment periods are alternated. It will be understood that the period of treatment in cycling therapy may continue until the patient has achieved a complete response or a partial response, at which point the treatment may be stopped. Alternatively, the period of treatment in cycling therapy may continue until the patient has achieved a complete response or a partial response, at which point the period of treatment may continue for a particular number of cycles. In some embodiments, the length of the period of treatment may be a particular number of cycles, regardless of patient response. In some other embodiments, the length of the period of treatment may continue until the patient relapses.

In some embodiments, tazemetostat and the CDK4/6 inhibitor, AURK inhibitor, or ATR inhibitor are cyclically administered to a patient. Cycling therapy involves the administration of a first agent (e.g., a first prophylactic or therapeutic agent) for a period of time, followed by the administration of a second agent and/or third agent (e.g., a second and/or third prophylactic or therapeutic agent) for a period of time and repeating this sequential administration. Cycling therapy can reduce the development of resistance to one or more of the therapies, avoid or reduce the side effects of one of the therapies, and/or improve the efficacy of the treatment.

In some embodiments, tazemetostat is administered for a particular length of time prior to administration of the CDK4/6 inhibitor, AURK inhibitor, or ATR inhibitor. For example, in a 21-day cycle, tazemetostat may be administered on days 1 to 5, days 1 to 7, days 1 to 10, or days 1 to 14, and the CDK4/6 inhibitor, AURK inhibitor, or ATR inhibitor may be administered on days 6 to 21, days 8 to 21, days 11 to 21, or days 15 to 21. In other embodiments, the CDK4/6 inhibitor, AURK inhibitor, or ATR inhibitor is administered for a particular length of time prior to administration of tazemetostat. For example, in a 21-day cycle, the CDK4/6 inhibitor, AURK inhibitor, or ATR inhibitor may be administered on days 1 to 5, days 1 to 7, days 1 to 10, or days 1 to 14, and tazemetostat may be administered on days 6 to 21, days 8 to 21, days 11 to 21, or days 15 to 21.

In one embodiment, the administration is on a 21-day dose schedule in which a once daily dose of tazemetostat is administered beginning on day eight for seven days, followed by seven days of non-treatment, in combination with twice-daily administration of the CDK4/6 inhibitor for seven days followed by 14 days of non-treatment (e.g., tazemetostat is administered on days 8-14 and the CDK4/6 inhibitor is administered on days 1-7 of the 21-day schedule). In another embodiment, the administration is on a 21-day dose schedule in which a once daily dose of CDK4/6 inhibitor is administered beginning on day eight for seven days, followed by seven days of non-treatment, in combination with twice-daily administration of tazemetostat for seven days followed by 14 days of non-treatment (e.g., the CDK4/6 inhibitor is administered on days 8-14 and tazemetostat is administered on days 1-7 of the 21-day schedule).

In one embodiment, the administration is on a 21-day dose schedule in which a once daily dose of tazemetostat is administered beginning on day eight for seven days, followed by seven days of non-treatment, in combination with twice-daily administration of the AURK inhibitor for seven days followed by 14 days of non-treatment (e.g., tazemetostat is administered on days 8-14 and the AURK inhibitor is administered on days 1-7 of the 21-day schedule). In another embodiment, the administration is on a 21-day dose schedule in which a once daily dose of AURK inhibitor is administered beginning on day eight for seven days, followed by seven days of non-treatment, in combination with twice-daily administration of tazemetostat for seven days followed by 14 days of non-treatment (e.g., the AURK inhibitor is administered on days 8-14 and tazemetostat is administered on days 1-7 of the 21-day schedule).

In one embodiment, the administration is on a 21-day dose schedule in which a once daily dose of tazemetostat is administered beginning on day eight for seven days, followed by seven days of non-treatment, in combination with twice-daily administration of the ATR inhibitor for seven days followed by 14 days of non-treatment (e.g., tazemetostat is administered on days 8-14 and the ATR inhibitor is administered on days 1-7 of the 21-day schedule). In another embodiment, the administration is on a 21-day dose schedule in which a once daily dose of ATR inhibitor is administered beginning on day eight for seven days, followed by seven days of non-treatment, in combination with twice-daily administration of tazemetostat for seven days followed by 14 days of non-treatment (e.g., the ATR inhibitor is administered on days 8-14 and tazemetostat is administered on days 1-7 of the 21-day schedule).

In some embodiments, tazemetostat in combination with an AURK inhibitor, ATR inhibitor, or CDK4/6 inhibitor are each administered at a dose and schedule typically used for that agent during monotherapy. In other embodiments, tazemetostat and one of AURK inhibitor, ATR inhibitor, or CDK4/6 inhibitor are administered concomitantly, one or both of the agents can advantageously be administered at a lower dose than typically administered when the agent is used during monotherapy, such that the dose falls below the threshold that an adverse side effect is elicited.

The therapeutically effective amounts or suitable dosages of tazemetostat, the AURK inhibitor, the ATR inhibitor and the CDK4/6 inhibitor in combination depends upon a number of factors, including the nature of the severity of the condition to be treated, the particular inhibitor, the route of administration and the age, weight, general health, and response of the individual patient. In certain embodiments, the suitable dose level is one that achieves a therapeutic response as measured by tumor regression or other standard measures of disease progression, progression free survival, or overall survival. In other embodiments, the suitable dose level is one that achieves this therapeutic response and also minimizes any side effects associated with the administration of the therapeutic agent.

Suitable daily dosages of ATR inhibitors can generally range, in single or divided or multiple doses, from about 10% to about 120% of the maximum tolerated dose as a single agent. In certain embodiments, the suitable dosages of ATR inhibitors are from about 20% to about 100% of the maximum tolerated dose as a single agent. In other embodiments, the suitable dosages of ATR inhibitors are from about 25% to about 90% of the maximum tolerated dose as a single agent. In some embodiments, the suitable dosages of ATR inhibitors are from about 30% to about 80% of the maximum tolerated dose as a single agent. In other embodiments, the suitable dosages of ATR inhibitors are from about 40% to about 75% of the maximum tolerated dose as a single agent. In some embodiments, the suitable dosages of ATR inhibitors are from about 45% to about 60% of the maximum tolerated dose as a single agent. In other embodiments, suitable dosages of ATR inhibitors are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of AURK inhibitors can generally range, in single or divided or multiple doses, from about 10% to about 120% of the maximum tolerated dose as a single agent. In certain embodiments, the suitable dosages of AURK inhibitors are from about 20% to about 100% of the maximum tolerated dose as a single agent. In other embodiments, the suitable dosages of AURK inhibitors are from about 25% to about 90% of the maximum tolerated dose as a single agent. In some embodiments, the suitable dosages of AURK inhibitors are from about 30% to about 80% of the maximum tolerated dose as a single agent. In other embodiments, the suitable dosages of AURK inhibitors are from about 40% to about 75% of the maximum tolerated dose as a single agent. In some embodiments, the suitable dosages of AURK inhibitors are from about 45% to about 60% of the maximum tolerated dose as a single agent. In other embodiments, suitable dosages of AURK inhibitors are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of CDK4/6 inhibitors can generally range, in single or divided or multiple doses, from about 10% to about 120% of the maximum tolerated dose as a single agent. In certain embodiments, the suitable dosages of CDK4/6 inhibitors are from about 20% to about 100% of the maximum tolerated dose as a single agent. In some other embodiments, the suitable dosages of CDK4/6 inhibitors are from about 25% to about 90% of the maximum tolerated dose as a single agent. In some other embodiments, the suitable dosages of CDK4/6 inhibitors are from about 30% to about 80% of the maximum tolerated dose as a single agent. In some other embodiments, the suitable dosages of CDK4/6 inhibitors are from about 40% to about 75% of the maximum tolerated dose as a single agent. In some other embodiments, the suitable dosages of CDK4/6 inhibitors are from about 45% to about 60% of the maximum tolerated dose as a single agent. In other embodiments, suitable dosages of CDK4/6 inhibitors are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of tazemetostat can generally range, in single or divided or multiple doses, from about 10% to about 120% of the maximum tolerated dose as a single agent. In certain embodiments, the suitable dosages of tazemetostat are from about 20% to about 100% of the maximum tolerated dose as a single agent. In some other embodiments, the suitable dosages of tazemetostat are from about 25% to about 90% of the maximum tolerated dose as a single agent. In some other embodiments, the suitable dosages of tazemetostat are from about 30% to about 80% of the maximum tolerated dose as a single agent. In some other embodiments, the suitable dosages of tazemetostat are from about 40% to about 75% of the maximum tolerated dose as a single agent. In some other embodiments, the suitable dosages of tazemetostat are from about 45% to about 60% of the maximum tolerated dose as a single agent. In other embodiments, suitable dosages of tazemetostat are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of tazemetostat, an AURK inhibitor, an ATR inhibitor, or a CDK4/6 inhibitor, sufficient for achieving a therapeutic or prophylactic effect, may range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of tazemetostat, AURK inhibitor, ATR inhibitor, or CDK4/6 inhibitor ranges from 0.001-10,000 micrograms per kg body weight. In one embodiment, tazemetostat, AURK inhibitor, ATR inhibitor, or CDK4/6 inhibitor concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of tazemetostat, an AURK inhibitor, an ATR inhibitor, or CDK4/6 inhibitor may be defined as a concentration of tazemetostat, the AURK inhibitor, the ATR inhibitor, or CDK4/6 inhibitor at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Kits of the Present Technology

The present disclosure provides kits for treating sarcoma comprising (a) tazemetostat, (b) at least one of a CDK4/CDK6 inhibitor, an AURK inhibitor, and ATR inhibitor, and (c) instructions for treating sarcomas. When simultaneous administration is contemplated, the kit may comprise tazemetostat and at least one of a CDK4/CDK6 inhibitor, an AURK inhibitor, and ATR inhibitor that has been formulated into a single pharmaceutical composition such as a tablet, or as separate pharmaceutical compositions. When tazemetostat and one or more of the CDK 4/6 inhibitors, AURK inhibitors, and ATR inhibitors disclosed herein are not administered simultaneously, the kit may comprise (a) tazemetostat, and (b) at least one of a CDK4/CDK6 inhibitor, an AURK inhibitor, and ATR inhibitor that has been formulated as separate pharmaceutical compositions either in a single package, or in separate packages.

Additionally or alternatively, in some embodiments, the kits further comprise at least one chemotherapeutic agent and/or at least one immunotherapeutic agent that are useful for treating sarcoma. Examples of such chemotherapeutic agents include abraxane, capecitabine, erlotinib, fluorouracil (5-FU), gemcitabine, irinotecan, leucovorin, nab-paclitaxel, cisplatin, irinotecan, docetaxel, oxaliplatin, tipifarnib, everolimus, sunitinib, dovitinib, ruxolitinib, pegylated-hyaluronidase, pemetrexed, folinic acid, paclitaxel, MK2206, GDC-0449, IPI-926, gamma secretase/R04929097, M402, and LY293111. Examples of such immunotherapeutic agents include immune checkpoint inhibitors (e.g., antibodies targeting CTLA-4, PD-1, PD-L1), ipilimumab, 90Y-Clivatuzumab tetraxetan, pembrolizumab, nivolumab, trastuzumab, cixutumumab, ganitumab, demcizumab, cetuximab, nimotuzumab, dalotuzumab, sipuleucel-T, CRS-207, and GVAX.

The kits may further comprise pharmaceutically acceptable excipients, diluents, or carriers that are compatible with one or more kit components described herein. Optionally, the above described components of the kits of the present technology are packed in suitable containers and labeled for the treatment of sarcoma. Examples of sarcoma include, but are not limited to rhabdoid sarcoma or epithelioid sarcoma. In some embodiments, the sarcoma comprises a deficiency in a BAF chromatin remodeling complex component or SMARCB1. Additionally or alternatively, in some embodiments, the subject comprises an acquired mutation in EZH2, CDKN2A B, CDKN1A, ANKRD11 or RB1. The kits may optionally include instructions customarily included in commercial packages of therapeutic products, that contain information about, for example, the indications, usage, dosage, manufacture, administration, contraindications and/or warnings concerning the use of such therapeutic products.

### EXAMPLES

The present technology is further illustrated by the following Examples, which should not be construed as limiting in any way.

### Example 1: Materials and Methods

### EZH2 Mutant Plasmids

The EZH2^{Y666N} mutation detected in the clinical trial patient refers to amino acid numbering in isoform 2 of the protein. For consistency of nomenclature, all engineered mutations use numbering referring to isoform 2 (Uniprot ID: Q15190-2), although isoform 1 was expressed in cells for this study. Plasmids containing wild-type EZH2 (EZH2^{WT}) and catalytically inactive triple mutant (F672I, H694A, R732K, referred to as EZH2^{CatMut}) plasmids were provided in the doxycycline-inducible pINDUCER20 vector (75). The plasmids contain human EZH2 tagged N-terminally with a FLAG-Avi tag.

The Y666N mutation was engineered in both the EZH2′T and EZH2^{CatMut} plasmids to yield EZH2^{Y666N} and EZH2^{QuadMut}, respectively. The mutation was introduced by site-directed mutagenesis using the QuikChange Lightning Kit (Agilent) per manufacturer's instructions (mutagenesis primers: 5′-GCAAAGTGTACGACAAGAACATGTGCAGCTTTCTG-3′ (SEQ ID NO: 1) and 5′-CAGAAAGCTGCACATGTTCTTGTCGTACACTTTGC-3′ (SEQ ID NO: 2)) to engineer a TAC to AAC codon change. After mutagenesis, PCR products were transformed into Stbl3 E. coli cells and expanded at 30° C. Correct plasmid sequences were confirmed by Sanger sequencing (Eton). The sequencing primers used are: F1: 5′-GGACAGCAGAGATCCAGTTTG-3′ (SEQ ID NO: 3); R1: 5′-GGTCCGTTCCAGGATCTTCT-3′ (SEQ ID NO: 4); F2: 5′-TCCAGTGTGGTGGAATTCTG-3′ (SEQ ID NO: 5); R2: 5′-TATCGCTGGGGAACTTTCTG-3′ (SEQ ID NO: 6); F3: 5′-TGCTGCACAACATCCCTTAC-3′ (SEQ ID NO: 7); R3: 5′-TGCTGGTTTCGTCCTTCTTT-3′ (SEQ ID NO: 8); F4: 5′-CCTACAAGCGGAAGAACACC-3′ (SEQ ID NO: 9); R4: 5′-GTTCTTGCTGTCCCAGTGGT-3′ (SEQ ID NO: 10); F5: 5′-CTGAAGAAGGATGGCAGCTC-3′ (SEQ ID NO: 11); R5: 5′-CTTGGGTGGGTTACTCCAGA-3′ (SEQ ID NO: 12)

### RB1 Knockout

G401 cells with RB1 mutations were engineered by Synthego. Briefly, a single guide RNA targeting exon 2 of RB1 was used (AGAGAGAGCUUGGUUAACUU (SEQ ID NO: 13)). Ribonucleprotein containing spCas9 and sgRNA was transfected into G401 cells by electroporation. The target site was then PCR-amplified and Sanger sequenced to ensure homozygous indels (PCR and sequencing primers: Forward-CACTGTGTGGTATCCTTATTTTGGA (SEQ ID NO: 14), Reverse-AGGTAAATTTCCTCTGGGTAATGGA (SEQ ID NO: 15), with the forward primer used for sequencing). The cells were then single-cell cloned and re-verified by Sanger sequencing. Loss of the RB1 protein was confirmed by Western blot.

### Cell Cycle Analysis

G401 cells were plated on Day 0 and treated for 10 days with 1 μm tazemetostat or equivalent volume of DMSO, with drug and media replaced on Days 4 and 7. On Day 11, cells were pulsed with EdU for 1 hour. Cells were then harvested and processed for flow cytometry using the manufacturer's protocol (Click-iT, Invitrogen). Briefly, cells were washed with PBS with 1% BSA, permeabilized, and incubated with AlexaFluor647 for 30 minutes. DNA content was measured using propidium iodide (0.05 μg/μL). Cells were analyzed on a CytoFLEX LX (Beckman Coulter).

### Patient Tumor Samples

Patient tumor and matched normal blood samples were obtained from patients at Memorial Sloan Kettering Cancer Center (MSKCC) enrolled in the TAZ clinical trial (13). All patients provided informed consent for this study under the Institutional Review Board approved research protocol 12-245. Patient tumors were classified into “Response” or “Progression” groups based on RECIST 1.1 criteria (76). “Response” included tumors exhibiting a complete response, partial response, or stable disease. All other tumors were classified under the “Progression” group. This cohort includes both tumor samples that underwent targeted sequencing with MSK-IMPACT (16) as part of their clinical care at MSKCC as well as archived tumors that were analyzed for this study. For genomic analysis, DNA was extracted from either flash frozen tumor samples or formalin-fixed, paraffin-embedded (FFPE) blocks or slides and samples were processed using the IMPACT468 or IPACT505 panels depending on the time of their sequencing (16). The detected mutations and copy number alterations were obtained from cBioPortal (77, 78). Oncoprints were generated using Oncoprinter (cBioPortal).

For transcriptomic analysis, archived frozen tumor samples were weighed and up to 20-30 mg were homogenized in RLT buffer, followed by extraction using the AllPrep DNA/RNA Mini Kit (QIAGEN catalog 80204) according to the manufacturer's instructions. RNA was eluted in 13 μL nuclease-free water. After RiboGreen quantification and quality control by Agilent BioAnalyzer, 1 μg of total RNA with DV200 percentages varying from 78% to 100% underwent ribosomal depletion and library preparation using the TruSeq Stranded Total RNA LT Kit (Illumina catalog RS-122-1202) according to instructions provided by the manufacturer with 8 cycles of PCR. Samples were barcoded and sequenced using NovaSeq 6000 in a PE150 mode, with the NovaSeq 6000 S4 Reagent Kit (Illumina). On average, 84 million paired reads were generated per sample and 70% of the data mapped to mRNA.

### Targeted Sequencing of Cell Lines

To assess for the presence of somatic mutations in MRT and ES cell lines, DNA was extracted using the PureLink Genomic DNA Minikit (Invitrogen) and processed using the IMPACT505 panel as above. Due to the lack of matched normal tissue for cell lines, copy number alterations were detected using a custom algorithm using circular binary segmentation (79) implemented by the MSK Bioinformatics core. Code is available on github at: https://github.com/kentsisresearchgroup/seqCNA_tazemetostat_resistance.

### Data Availability

RNA-seq data from G401 cells can be found at the Gene Expression Omnibus (GEO) repository, accession number GSE213845. RNA-seq data from patient tumor samples has been deposited to the Database of Genotypes and Phenotypes (dbGaP), accession number phs003188.v1.p1. Additional supplementary data files and computational analysis scripts are available at Zenodo (10.5281/zenodo.7595037).

### Cell Culture

All cell lines were obtained from the American Type Culture Collection if not otherwise specified. ES1 and ES2 cells were generated and kindly provided by Nadia Zaffaroni. EPI544 cells were obtained from the MD Andersen Cancer Center Cytogenetics and Cell Authentication Core. Rhabdoid tumor cell lines KP-MRT-NS, KP-MRT-RY, and MP-MRT-AN were kindly provided by Yasumichi Kuwahara and Hajime Hosoi. The identity of all cell lines was verified by STR analysis. Absence of Mycoplasma contamination was determined using the MycoAlert kit according to manufacturer's instruction (Lonza). Cell lines were cultured in 5% CO₂ in a humidified atmosphere in 37° C. All media were obtained from Corning and supplemented with 10% fetal bovine serum (FBS), 1% L-glutamine, and 100 U/mL penicillin and 100 μg/mL streptomycin (Gibco). RPE, G401, A204, ES1, ES2, and VAESBJ cells were cultured in Dulbecco's Modified Eagle Medium (DMEM). TTC642, TM8716, MP-MRT-AN, KP-MRT-NS, and KP-MRT-RY cells were cultured in Roswell Park Memorial Institute (RPMI) medium. EPI544 cells were cultured in DMEM/F12 medium.

### Western Blotting

To assess protein expression by Western immunoblotting, pellets of 1 million cells were prepared and washed once in cold PBS. Cells were resuspended in 100-130 μL of RIPA lysis buffer (50 mM Tris-HCl, pH 8.0, 150 mM NaCl, 1.0% NP-40, 0.5% sodium deoxycholate, 0.1% sodium dodecyl sulfate) and incubated on ice for 10 minutes. Cell suspensions were then disrupted using a Covaris S220 adaptive focused sonicator for 5 minutes (peak incident power: 35W, duty factor: 10%, 200 cycles/burst) at 4° C. Lysates were cleared by centrifugation at 18,000 g for 15 min at 4° C. Protein concentration was assayed using the DC Protein Assay (Bio-Rad) and 15-35 μg whole cell extract was used per sample. Samples were boiled at 95° C. in Laemmli buffer (Bio-Rad) with 40 mM DTT and resolved using sodium dodecyl sulfate-polyacrylamide gel electrophoresis. Proteins were transferred to Immobilon FL PVDF membranes (Millipore), and membranes were blocked using Intercept Blocking buffer (Li-Cor). Primary antibodies used were: anti-EZH2 (Cell Signaling Technology, 5246) at 1:1,000, anti-RB1 (Cell Signaling Technology, 9309) at 1:250, anti-H3K27me3 (Cell Signaling Technology, 9733) at 1:500, anti-p16 (Abcam, ab108349) at 1:500, anti-CCNA2 (Santa Cruz, sc-271682) at 1:100, anti-PRICKLE1 (Santa Cruz, sc-393034) at 1:100, anti-SMARCB1 (BD Biosciences, 612110) at 1:500, anti-RPA32 pT21 (abcam, ab109394) at 1:2,000, anti-RPA32 pS4/pS8 (ThermoFisher, A300-245A) at 1:2,000, anti-pCHK1 S296 (Cell Signaling Technology, 90178) at 1:250, anti-MYCN (Cell Signaling Technology, 9405) at 1:250, anti-Actin (Cell Signaling Technology, 4970 and 3700) at 1:5,000. Blotted membranes were visualized using goat secondary antibodies conjugated to IRDye 680RD or IRDye 800CW (Li-Cor, 926-68071 and 926-32210) at 1:15,000 and the Odyssey CLx fluorescence scanner, according to manufacturer's instructions (Li-Cor). Image analysis was done using the Li-Cor Image Studio software (version 4).

### Lentivirus Production

Lentivirus production was carried out as described previously (1). Briefly, HEK293T cells were transfected using TransIT-LT1 using a 2:1:1 ratio of the lentiviral vector and psPAX2 and pMD2.G packaging plasmids, according to manufacturer's instructions (Mirus). Viral supernatant was collected at 48 and 72 hours post-transfection, pooled, filtered and stored in aliquots at −80° C. G401 cells were transduced at a multiplicity of infection (MOI) of 0.3. Transduced cells were selected for 7 days with G418 sulfate (ThermoFisher) at 1 mg/mL. Single-cell clones were then isolated and expanded. Inducible EZH2 expression was confirmed by Western blotting against EZH2.

### Cell Viability Testing

Drugs used for in vitro treatment were supplied by Selleckchem (TAZ; S7128, Elimusertib; S9864, abemaciclib; LY2835219, palbociclib; S1116, seleciclib; S1153, alisertib; S1133, barasertib; S1147, camptothecin, S1288).

The effects of RB1 loss or EZH2 mutation on TAZ susceptibility was assessed over 14 days. Cells were plated in 96-well microplates at equal densities and treated with 10 μM TAZ or equivalent volume of DMSO on Day 0. Drug and media were replaced on Days 4, 7, and 11. CellTiter-Glo assays were performed on Day 14, with luminescence readings taken using an automated fluorescence plate reader (Tecan). CellTiter-Glo reagent was freshly reconstituted on the day of measurement and added in a 1:1 proportion to cell media. A similar protocol was used for all other cell viability experiments, with treatment times indicated in the relevant figure legends. Cell line doubling time was determined by measuring cell viability every 24 hours over the course of 4 days, and fitting the cell viability to a two-parameter exponential curve. For combination treatment with TAZ and elimusertib, we used a two-dimensional dose matrix design, treating the cells for 9 days. After the addition of cells, drugs were added using a pin tool (stainless steel pins with 50 nL slots, V&P Scientific) mounted onto a liquid handling robot (CyBio Well vario, Analytik Jena). For analysis of synergy, we used the synergyFinder package (2). Outliers due to pinning errors were excluded after manual examination.

### Cell Line RNA-Sequencing

G401 cells with or without RB1 loss were plated and treated with 10 μM TAZ or equivalent volume of DMSO on Day 0. Drug and media were replaced on Days 4 and 8. Cells were harvested on Day 11 and RNA was isolated using RNeasy Mini, according to manufacturer's instructions (Qiagen). After RiboGreen quantification and quality control by Agilent BioAnalyzer, 149-500 ng of total RNA underwent Poly(A) selection and TruSeq library preparation according to instructions provided by Illumina (TruSeq Stranded mRNA LT Kit, catalog RS-122-2102), with 8 cycles of PCR. Samples were barcoded and sequenced using a HiSeq 4000 instrument using 50 bp/50 bp paired end mode, using the HiSeq 3000/4000 SBS Kit (Illumina). An average of 42 million paired reads was generated per sample. Ribosomal reads represented less than 0.03% of the total reads generated and proportion of mRNA bases averaged 74%.

### Analysis of RNA-Seq Data

For RNA-seq analysis of G401 cell lines, read adaptors were trimmed and quality filtered using ‘trim_galore’ (v0.4.4_dev) and mapped to GRCh38/hg19 reference genome using STAR v2.6.0a with default parameters (3). Read counts tables were generated using HTSeq (4). Normalization was performed using DESeq2 using the default parameters (5).

For RNA-seq analysis of patient tumor samples, read adaptors were trimmed and quality filtered using ‘trim_galore’ and mapped to GRCh38/hg19 reference genome using STAR v2.7.9 with default parameters (3). Read count tables were generated using HTSeq v0.11.3 (4). Bam files were sorted by name using ‘samtools’ and alignment quality was assessed using ‘qualimap’ v2.2.2. Normalization was performed using DESeq2 v1.34.0 using the default parameters (5). To assess gene expression changes between TAZ-sensitive and TAZ-resistant tumors, samples in both categories were compared by two-tailed Student's t-test using ‘rowttests’ in R v4.1.3. Genes were filtered by p<0.05 and sorted by t-statistic. Heatmaps were then generated using ‘pheatmap.’ Genome browser tracks were visualized from bam files using Integrated Genomics Viewer v2.13.1.

### Gene Ontology Analysis

Genes significantly up- or down-regulated in TAZ-sensitive and TAZ-resistant tumors determined by two-tailed Student's t-test at p-value <0.5 were searched against the Gene Ontology database (DOI: 10.5281/zenodo.5725227 Downloaded 2021-11-16).

### Microscopy

Bright field microscopy was performed using an Evos FL Auto 2 imager at 10× magnification, with cells grown on plastic dishes. Immunofluorescence for γH2AX was performed on cells plated on Millicell EZ Slide glass slides (EMD Millipore), coated for 45 minutes with bovine plasma fibronectin (Millipore Sigma). After drug treatment, cells were washed once with PBS and fixed in 4% formaldehyde for 10 minutes at room temperature. Slides were then washed three times in PBS for 5 minutes, permeabilized for 15 minutes in 0.3% Triton X-100, washed again in PBS three times, and blocked with 5% goat serum (Millipore Sigma, G9023) in PBS for 1 hour at room temperature. Slides were incubated with mouse anti-γH2A.X primary antibody (Sigma-Aldrich, 05-636) at 1:500 in blocking buffer for 1 hour, washed three times in PBS, and incubated with goat anti-mouse secondary antibody conjugated to AlexaFluor555 (Invitrogen, A-21422) at 1:1,000. Cells were then counterstained with DAPI at 1:1,000 for 10 minutes and treated with ProLong Diamond Antifade Mountant with DAPI (Invitrogen, P36962) for 48 hours.

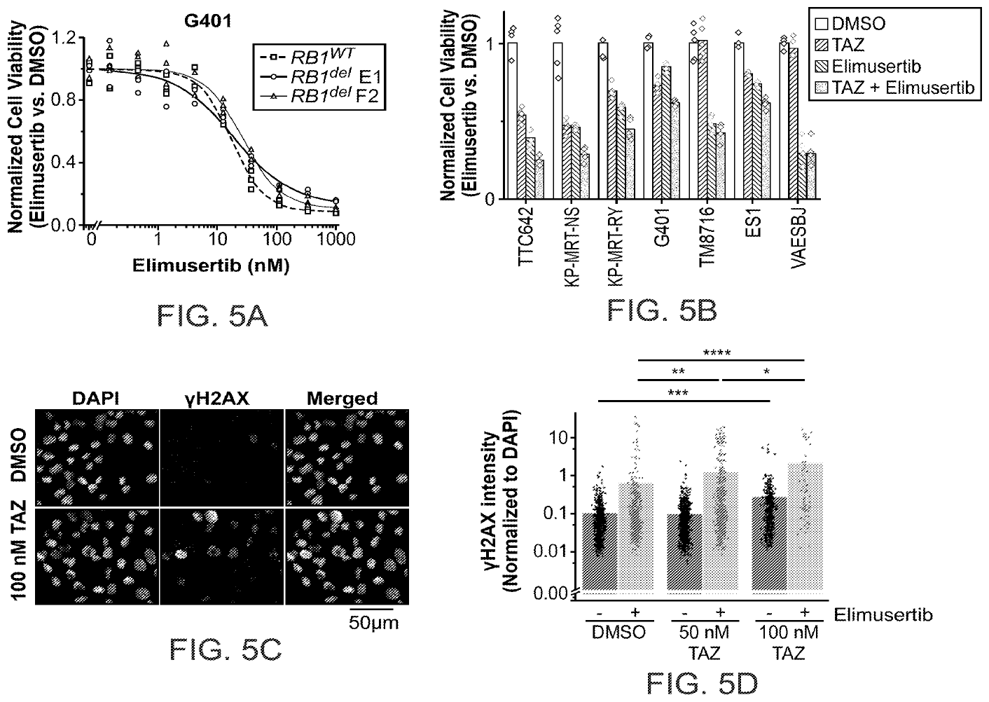

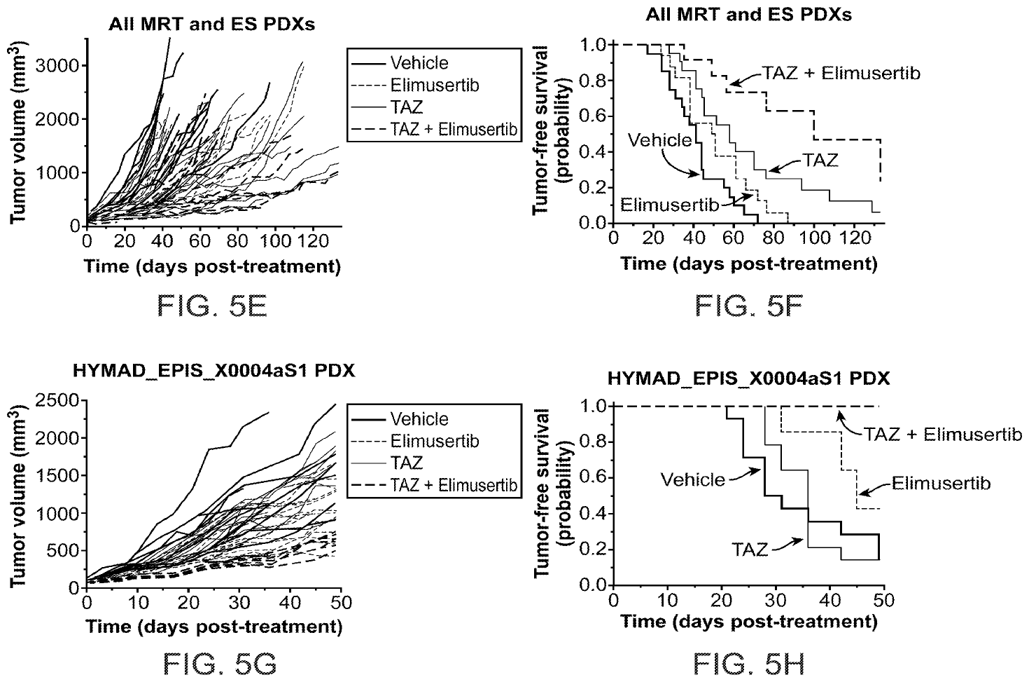

Images were acquired on a Leica SP5 confocal microscope in the upright configuration at 63× magnification. Images were then processed using a custom pipeline in CellProfiler (6). Per-cell integrated γH2A.X intensity was normalized against per-cell integrated DAPI intensity. Overlaid images in FIG. 5 were prepared using Fiji (7).

### Xenografts

All mouse experiments were carried out in accordance with institutionally approved animal use protocols. To generate PDXs, tumor specimens were collected under approved TRB protocol 14-091, immediately minced and mixed (50:50) with Matrigel (Corning, New York, NY) and implanted subcutaneously in the flank of 6-8 weeks-old female NOD.Cg-Prkdc^{scid} Il2rg^{tm1Wj1}/Szj (NSG) mice (Jackson Laboratory, Bar Harbor, ME), as described previously (8). Mice were monitored daily and PDX samples were serially transplanted three times before being deemed established. PDX tumor histology was confirmed by review of H&E slides and direct comparison to the corresponding patient tumor slides. PDX identity was further confirmed by MSK-IMPACT sequencing analysis.

Therapeutic studies used female and male NSG mice obtained from the Jackson Laboratory. Xenografts were prepared as a single-cell suspensions, resuspended in Matrigel, and implanted subcutaneously into the right flank of 6-10 week old mice. 100 μL of tumor cell suspension was used for each mouse. Tumors were allowed to grow until they reached a volume of 100 mm³, at which point they were randomized into treatment groups without blinding. Drugs were prepared using the following formulations: Tazemetostat was dissolved at 25 mg/mL in 5% DMSO, 40% PEG 300, 5% Tween 80, and 50% water. Elimusertib was dissolved at 5 mg/mL in 10% DMSO, 40% PEG 300, 5% Tween 80, and 45% water using a sonicator. Barasertib was dissolved at 2.5 mg/mL in 5% DMSO, 40% PEG 300, 5% Tween 80, and 50% water. Drugs were reconstituted daily. The following drug doses and schedules were used: TAZ was dosed at 250 mg/kg twice daily by oral gavage, 7 days per week. Barasertib was dosed at 25 mg/kg once daily by intraperitoneal injection using 3 days on and 4 days off cycle. Elimusertib was dosed at 40 mg/kg twice daily by oral gavage using 2 days on and 12 days off cycle. Caliper tumor measurements were taken twice weekly. Tumor volumes were calculated using the formula Volume=(7c/6)×length×width². Tumor growth analysis was performed using the Vardi U-test (9), as implemented in the clinfun R package using the aucVardiTest function. Tumor-free survival analysis was calculated using OriginPro (Microcal) by the Kaplan-Meier method, using the log-rank test.

### Example 2: Patient Tumor Sequencing Reveals Diverse Resistance Mutations

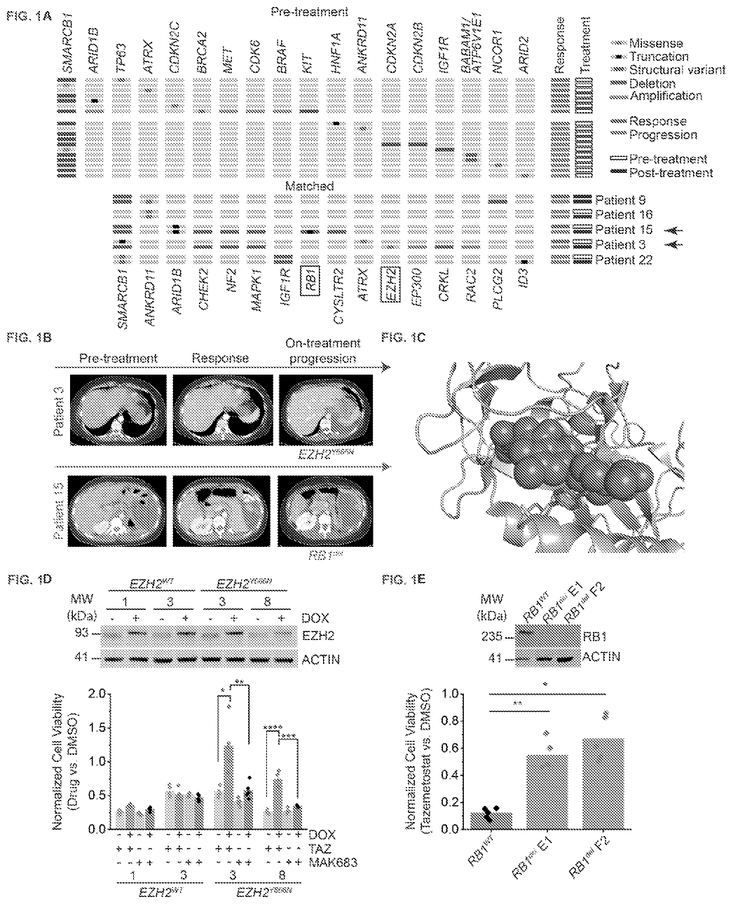

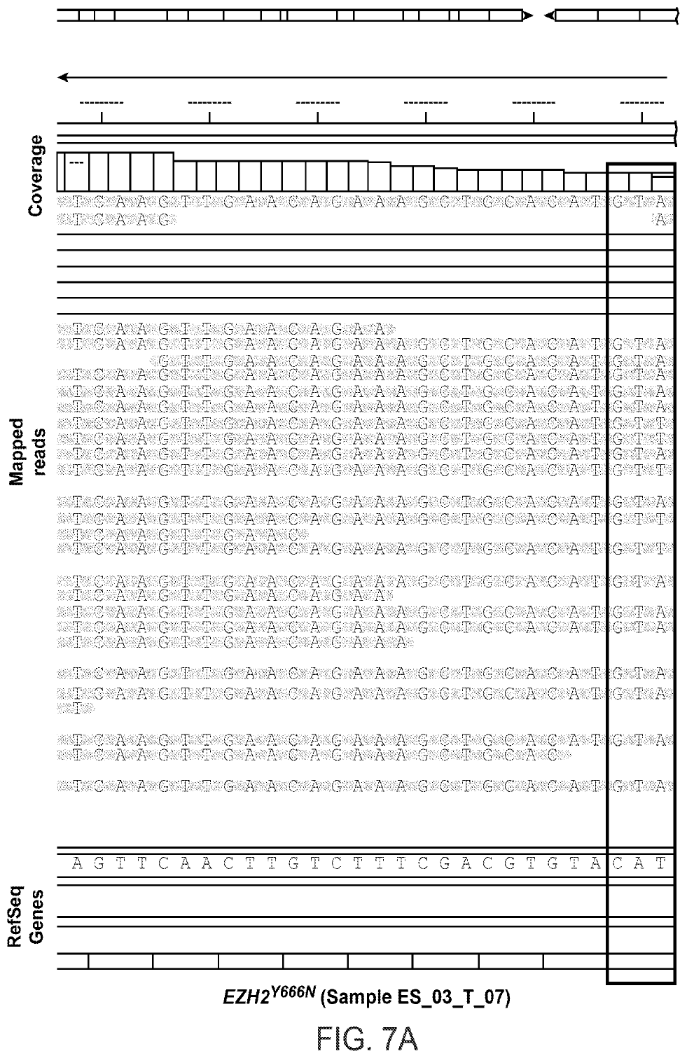

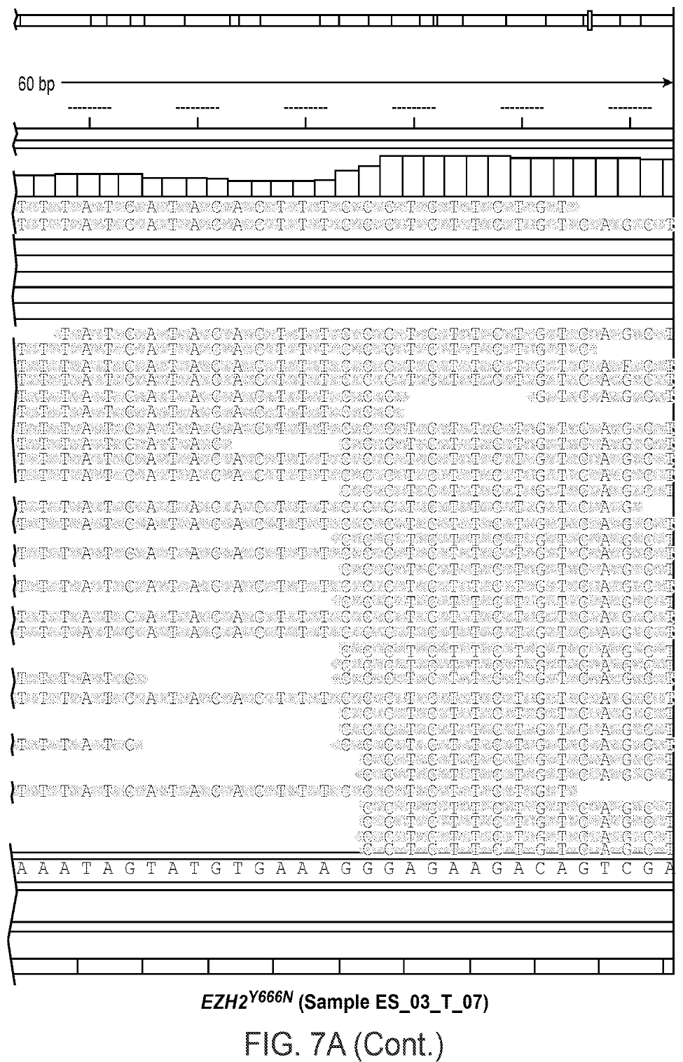

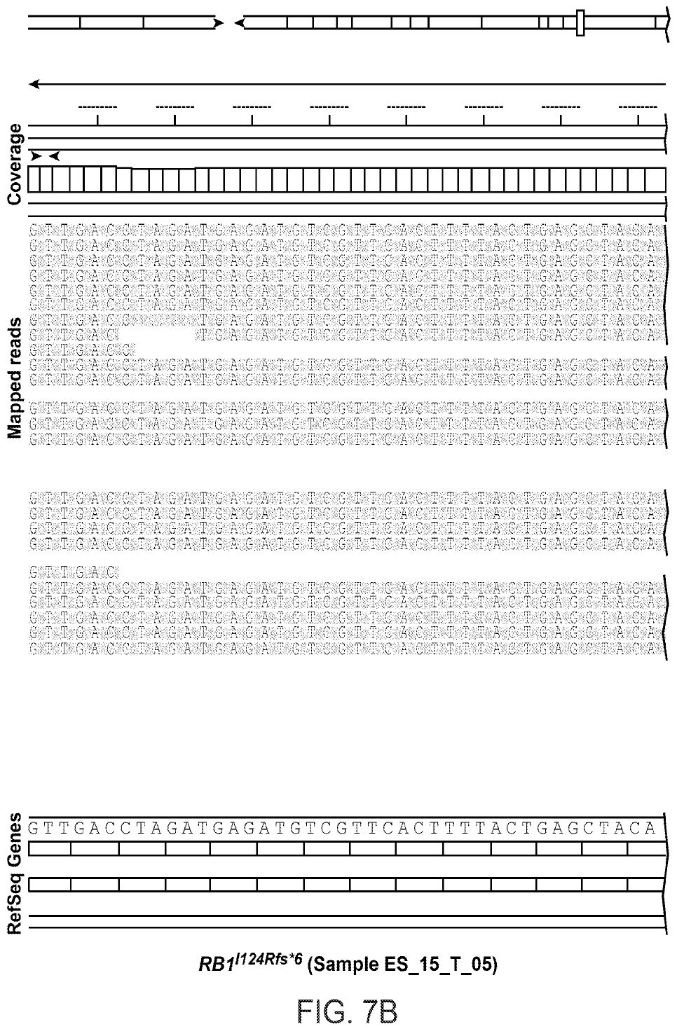

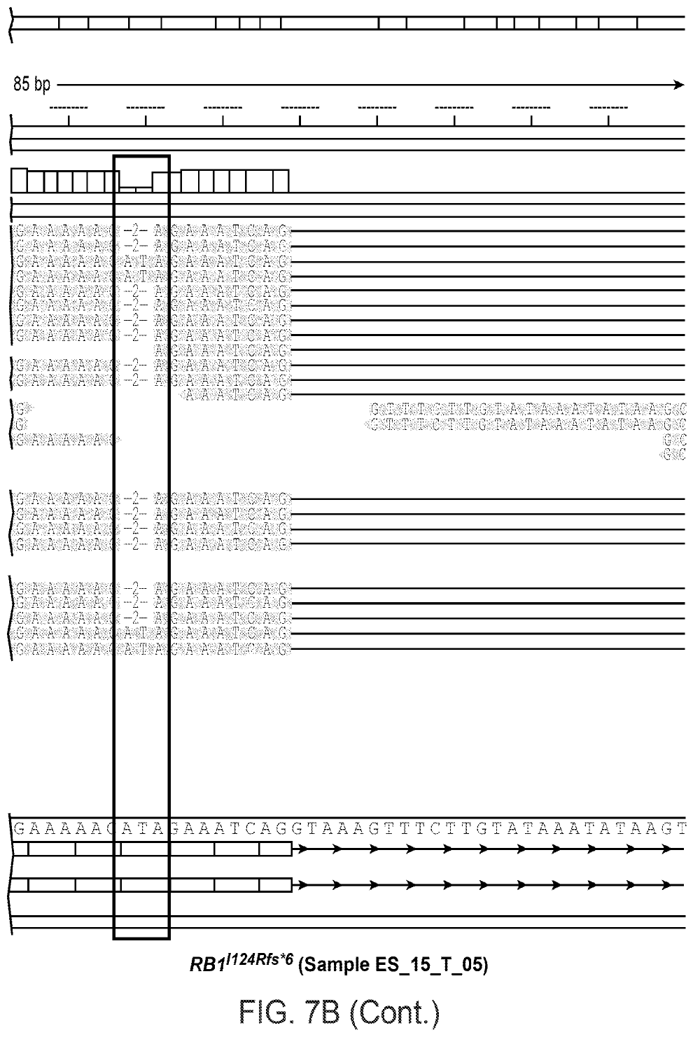

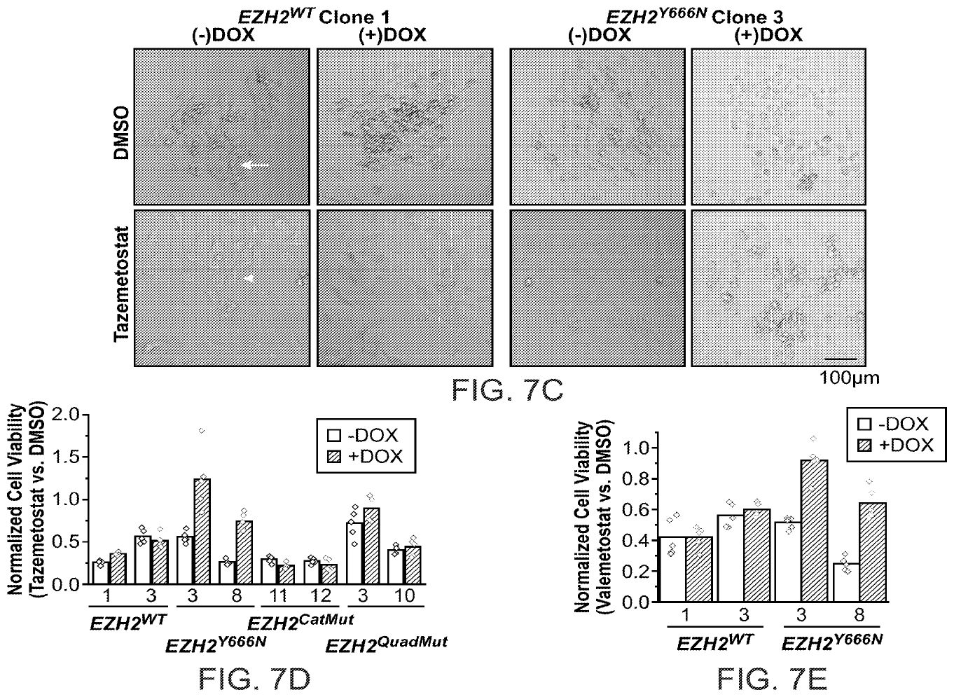

To identify mutations associated with clinical resistance to TAZ, we performed targeted gene sequencing of patient tumors using MSK-IMPACT, which is based on a panel of over 500 genes recurrently mutated in diverse cancer types (16). We analyzed 33 tumor specimens from 20 patients treated as part of the recent TAZ clinical trial (13), and identified somatic tumor mutations in matched pre- and post-treatment specimens. We found distinct sets of somatic mutations in responding and non-responding tumors, with nearly all mutations, apart from SMARCB1 loss itself, being exclusive to either TAZ-responsive or TAZ-resistant tumors (FIG. 1A, top panel). Strikingly, we observed two tumors which initially responded to TAZ based on radiographic imaging but later developed clinical resistance (FIG. 1A, bottom panel, FIG. 1B). Targeted sequencing of the resistant tumors revealed two newly acquired somatic mutations: homozygous missense mutation of EZH2 (EZH2^{Y666N}) in the Patient 3 tumor specimen, and biallelic loss of function mutation of RB1, including a hemizygous deletion and a frame shift mutation (RB1^{del}) in the remaining allele, in the Patient 15 tumor specimen. We confirmed both mutations using RNA-seq of the respective tumor specimens (FIGS. 7A-7B). Since one mutation affected EZH2 directly, and the other involved the known EZH2 target RB1 (17), we hypothesized that both mutations were responsible for TAZ resistance in their respective patients.

First, we investigated the EZH2^{Y66N} mutation. Past studies using forward genetic screens in lymphoma cell lines have identified putative resistance mutations within both the N-terminal D1 domain and the catalytic SET domain of EZH2, both of which are predicted to interact with S-adenosyl methionine (SAM)-competitive EZH2 inhibitors such as TAZ (18, 19). One such SET domain mutation previously identified in cell lines is EZH2^{Y661D}, with Y661 corresponding to Y666 in isoform 2 of EZH2, the isoform referred to in this study, and thus concordant with the mutation we observed in the Patient 3 tumor (18).

Based on the atomic resolution structure of a pyridone-based EZH2 inhibitor bound to the Anolis carolinensis EZH2 (PDB 5IJ7) (20), we reasoned that residue Y666 in human EZH2 may form a critical part of the TAZ binding site and that its mutation can prevent TAZ from binding to the SET domain (FIG. 1C). To test EZH2^{Y666N} as a resistance allele, we expressed doxycycline-inducible EZH2^{Y666N} in SMARCB1-deficient G401 rhabdoid tumor cells. We observed that EZH2^{Y666N} expressing clones are resistant to TAZ as compared to cells expressing equal levels of wild-type EZH2 by assessing both cell viability (FIG. 1D) and cell morphology (FIG. 7C). The resistance phenotype of EZH2^{Y666N}-expressing cells depends on the intact catalytic activity of the SET domain, as a compound mutant combining the catalytically inactive triple mutant EZH2^{F672I,H694A,R732K} (EZH2^{CatMut}) with the Y666N mutation, termed EZH2^{QuadMut}, did not confer resistance to TAZ (FIG. 7D). We also observed that EZH2^{Y666N} confers resistance to the dual EZH1/2 inhibitor valemetostat (21), consistent with putative resistance to SAM-competitive, pyridone-based EZH2 inhibitors (FIG. 7E). This also suggests that combined inhibition of EZH2 and EZH1 may not overcome this type of acquired EZH2 inhibitor resistance.

Previous studies found that lymphoma cells resistant to EZH2 inhibitors remained susceptible to the inhibition of the non-enzymatic PRC2 subunit EED (22), including those with mutations in the EZH2 SET domain (EZH2^{C663Y} and EZH2^{Y726F}) and the D1 domain (19, 23). We therefore hypothesized that TAZ resistance conferred by EZH2^{Y666N} could be overcome by PRC2 inhibitors that do not bind to EZH2. Indeed, we found that the allosteric EED inhibitor MAK683 overcomes EZH2^{Y666N}-mediated resistance (24), demonstrating that these cells remain generally susceptible to PRC2 inhibition (FIG. 1D). EED inhibition may thus be an effective strategy to overcome acquired TAZ resistance mutations in EZH2.

### Example 3: RB1 Loss Allows Escape from Cell Cycle Arrest Despite Effective EZH2 Inhibition

Past work has shown that EZH2 is a direct target of repression by RB1/E2F (17, 25). This suggests that acquired RB1 loss observed upon TAZ treatment may confer resistance to EZH2 inhibition by increasing EZH2 expression. To test RB1^{del} as a TAZ resistance allele, we used CRISPR/Cas9 genome editing to generate biallelic RB1^{del} mutations in G401 cells, as compared to isogenic RB1-wild type control cells produced by targeting the safe harbor locus AAVS1. We confirmed absence of RB1 protein expression in two independent clones using Western blotting, and found that RB1^{del} cells were indeed resistant to TAZ (FIG. 1E).

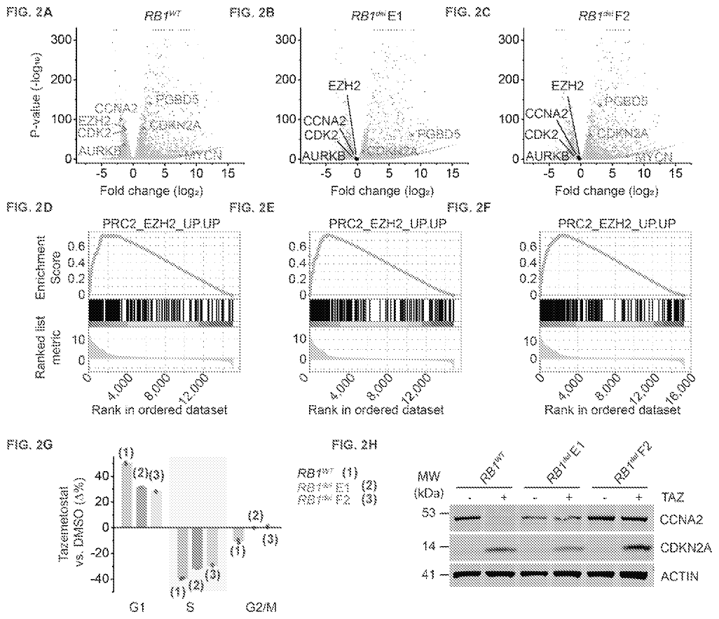

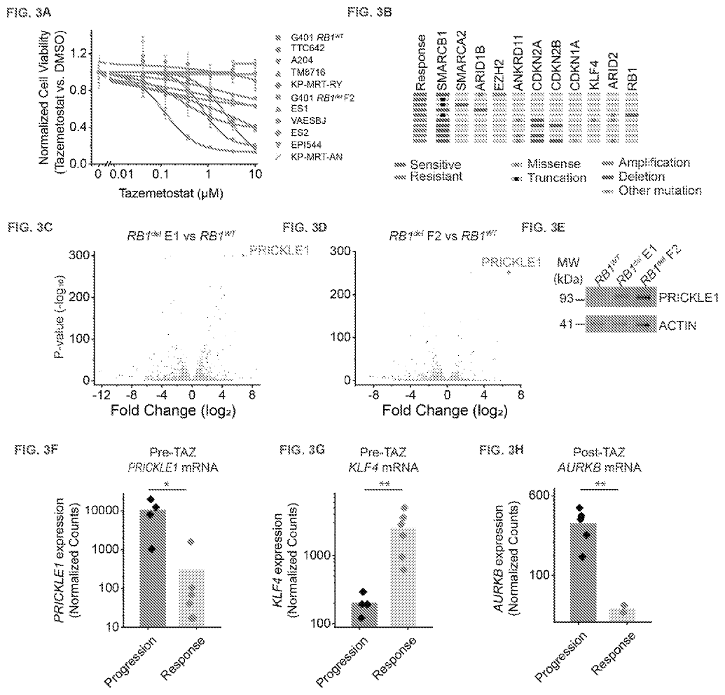

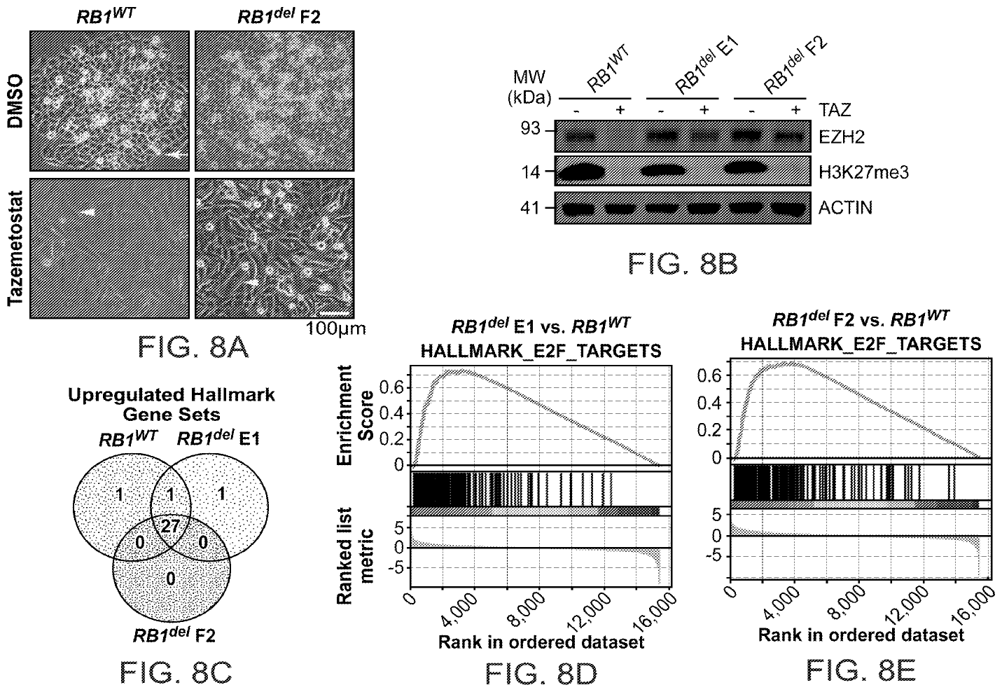

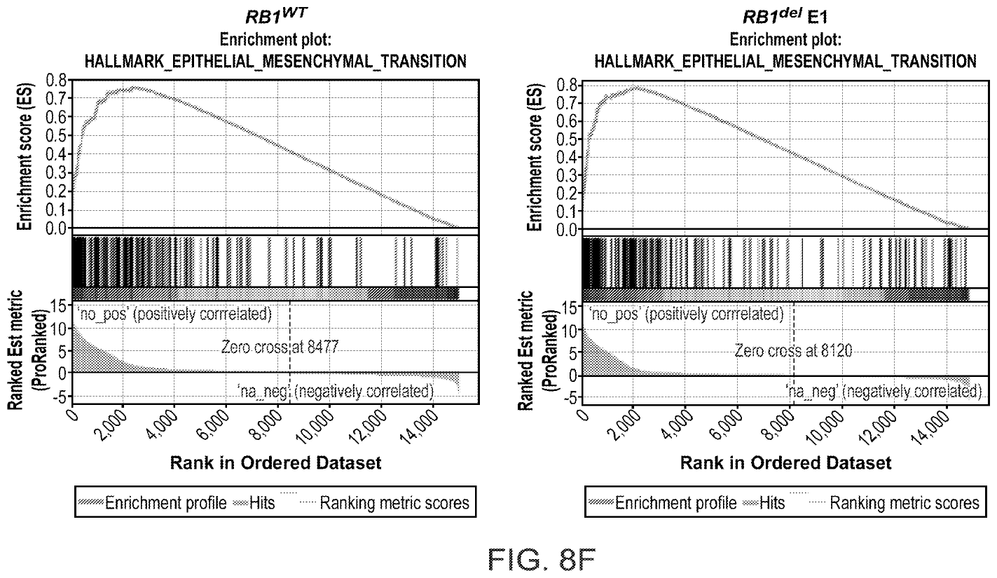

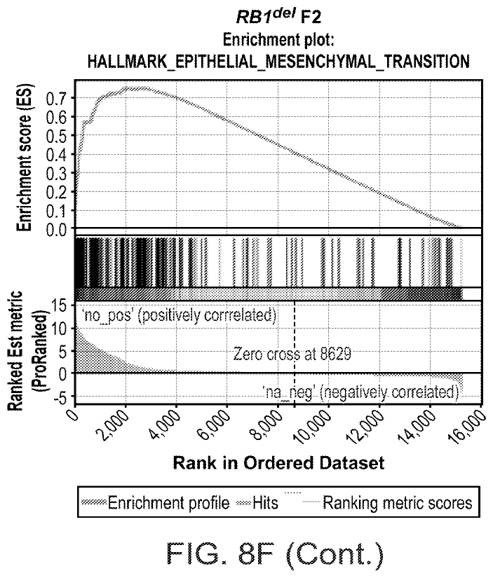

Despite EZH2 being a known target gene of RB1/E2F, we were surprised to observe that RB1^{del} G401 cells showed similar morphological changes upon TAZ treatment (FIG. 8A) to those previously reported for TAZ-treated RB1^{WT} G401 cells (7). To define the effects of TAZ on RB1^{del} cells more precisely, we performed RNA-seq of isogenic G401 RB1^{del} and wildtype AAVS1-control cells, treated with either 10 μM TAZ or DMSO control for 11 days, based on an established treatment regimen to model EZH2 inhibition in vitro (7). We observed that EZH2 mRNA and protein levels remained high in TAZ-treated RB1^{del} cells, unlike in RB1^{WT} cells (FIGS. 2A-2C, 8B). However, EZH2 inhibition induced significant upregulation of hundreds of genes in both RB1^{WT} and RB1^{del} cells, including upregulation of known PRC2 target genes (FIGS. 2A-2F).

Importantly, trimethylation of the EZH2 substrate H3K27 was substantially reduced by TAZ regardless of RB1 status (FIG. 8B). This indicates that despite persistent EZH2 expression, EZH2 methyltransferase activity is effectively inhibited by TAZ despite RB1 loss. Gene set enrichment analysis (GSEA) showed that all three clones significantly upregulated multiple gene sets upon TAZ treatment (FIG. 8C). This included gene sets associated with cell differentiation such as epithelial-to-mesenchymal transition (EMT; FIG. 8F). This is reminiscent of recent observations that re-expression of SMARCB1 in G401 cells can lead to a mesenchymal chromatin state (26), and is consistent with the idea that PRC2 inhibition may allow BAF to re-activate a more developmentally normal gene expression state. Taken together, these findings indicate that RB1 loss-induced TAZ resistance is independent of EZH2.

In addition to EZH2, additional RB1/E2F target genes were also upregulated in TAZ-treated RB1^{del} cells compared to TAZ-treated RB1^{WT} control cells (FIGS. 8D-8E). Given the function of RB1 in the regulation of the G1/S cell cycle checkpoint, we hypothesized that RB1 loss could allow cells to escape TAZ-induced cell cycle arrest. Flow cytometry cell cycle analysis showed that G401 cells treated with 1 μM TAZ arrest at the G1/S checkpoint, as reported previously (7). However, RB1^{del} cells exhibited a significant reduction in the proportion of cells in G1 phase upon TAZ treatment (50% of TAZ-treated RB1^{WT} cells versus 31% and 28% for RB1^{del} E1 and F2 clones, respectively), with a corresponding increase of the proportion of cells remaining in S and G2/M phases (FIG. 2G). In agreement with this, we observed persistent mRNA expression of S/G2/M-phase-associated CCNA2, CDK2, and AURKB genes in RB1^{del} cells upon TAZ treatment (FIGS. 2A-2C). This is despite upregulation of the CDK4/6 inhibitor CDKN2A (p16), a known PRC2 target in MRT (5, 27, 28), upon TAZ treatment in both RB^{WT} and RB1^{del} cells (FIGS. 2A-2C). We confirmed persistent maintenance of S-phase cyclin A2 (CCNA2) protein levels despite p16 upregulation using Western blotting (FIG. 211). Together, these results show that RB1 loss is sufficient to evade TAZ-induced cell cycle arrest at the G1/S restriction point despite maintaining the expected global transcriptional response to EZH2 inhibition, including upregulation of cell cycle inhibitor genes.

### Example 4: Intact RB1/E2F Axis is Required for TAZ Susceptibility

The requirement for RB1 expression in the therapeutic response to TAZ suggests that an intact RB1/E2F axis may be a general requirement for effective EZH2 inhibitor therapy. This would predict that other genetic and epigenetic perturbations to the RB1/E2F axis, beyond RB1 loss itself, would similarly confer escape from TAZ-induced cell cycle arrest. Analysis of our TAZ clinical trial treatment cohort revealed one patient tumor specimen with primary resistance to TAZ with intact RB1 but inactivating mutations of both CDKN2A and CDKN2B (FIG. 1A), both of which are known to inhibit CDK4/6-mediated phosphorylation of RB1. Two additional specimens had missense mutations in ANKRD11: One tumor with primary resistance to TAZ and another which initially responded but later progressed on treatment, at which point a newly acquired ANKRD11 mutation was detected (FIG. 1A—Patients 9 and 16, respectively). ANKRD11 is a known TP53 cofactor and putative tumor suppressor that contributes to TP53-mediated expression of pan-CDK inhibitor CDKN1A (29-31). CDKN1A itself is also known to be a PRC2 target in tumors (32-34), although its role in the response to EZH2 inhibition in SMARCB1-deficient sarcomas is currently unknown. These results converge on the dysregulation of the RB1/E2F axis as a mechanism of evasion of TAZ-induced cell cycle arrest.

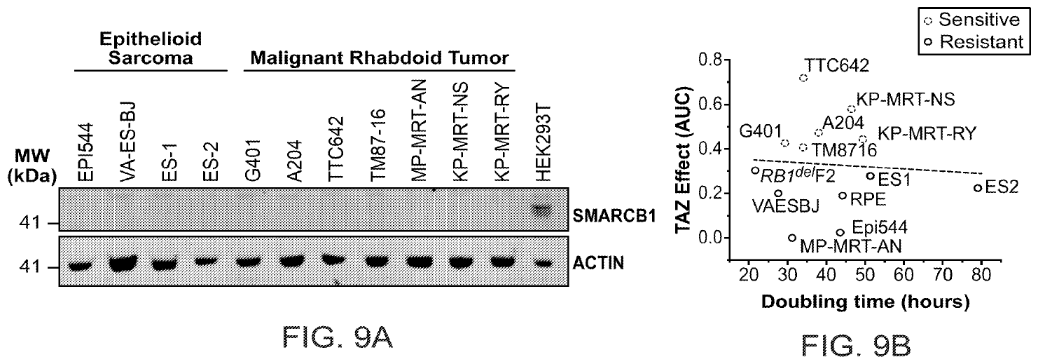

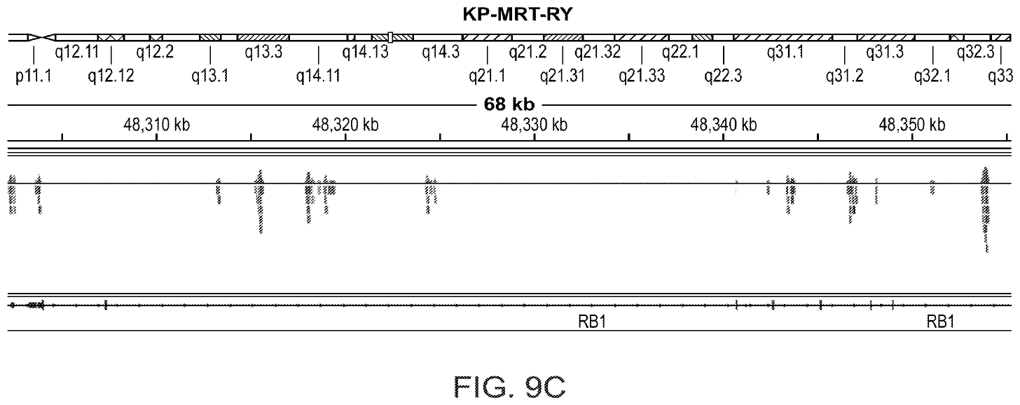

To investigate the functional determinants of tumor cell response to TAZ, we first analyzed the response to TAZ of seven MRT and four ES cell lines in which we confirmed loss of SMARCB1 protein expression in all ES and MRT cell lines using Western blotting, as compared to SMARCB1-expressing HEK293T cells (FIG. 9A). We classified each line as sensitive or resistant based on the area under the curve (AUC) of their TAZ dose responses (AUC>0.3 for sensitive G401, KP-MRT-NS, TTC642, A204, TM8716, KP-MRT-RY cell lines and AUC≤0.3 for resistant ES1, VAESBJ, ES2, EPI544, MP-MRT-AN cell lines; FIG. 3A). Given that TAZ treatment requires at least 4 days for the cellular reduction of methylated EZH2 substrates and at least 7 days for apparent antiproliferative effects in the rapidly-dividing G401 cell line (7), we confirmed that the apparent TAZ susceptibilities of these MRT and ES cell lines are not correlated with their proliferation rates (Pearson's r=−0.073 and p=0.81; FIG. 9B). In agreement with somatic mutations affecting the RB1/E2F axis associated with TAZ resistance in clinical tumor specimens (FIG. 1A), we found mutations of CDKN2A in 4 out of 5 TAZ-resistant cell lines, CDKN2B in 2 out of 5 resistant cell lines, CDKN1A in 1 out of 5, and ANKRD11 in 3 out of 5, as compared to no such mutations in any TAZ-sensitive MRT and ES cell lines (FIG. 3B). While our analysis detected reduction in copy number of RB1 in TAZ-sensitive KP-MRT-RY cells, manual inspection of sequencing reads within the RB1 gene revealed lack of homozygous deletion, with presumed retention of RB1 expression (FIG. 9C). Thus, mutations associated with the RB1/E2F axis are associated with resistance to TAZ in SMARCB1-deficient cell lines and patient tumors.

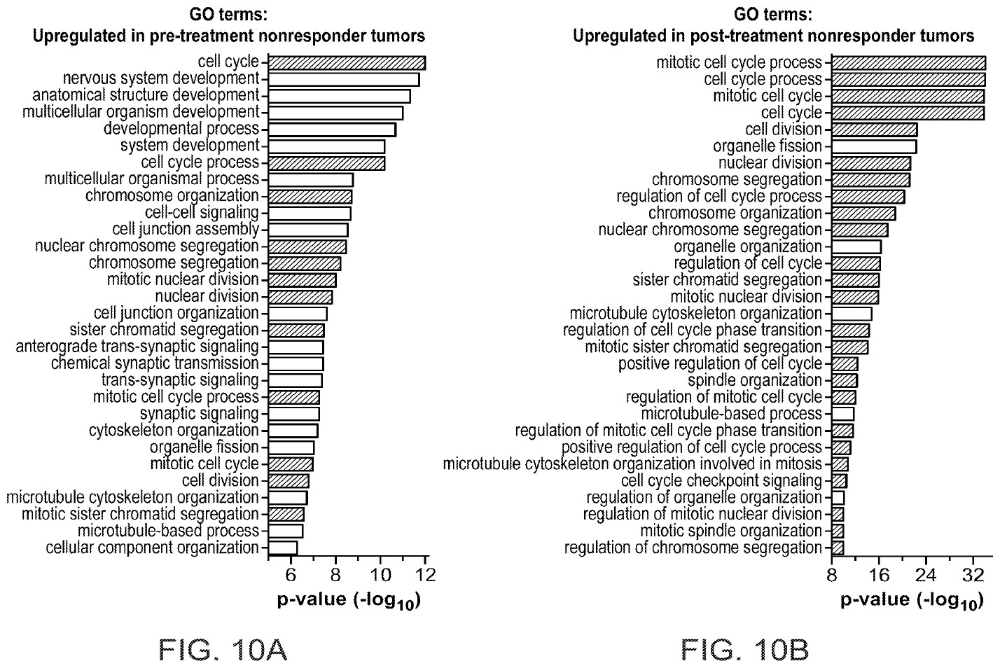

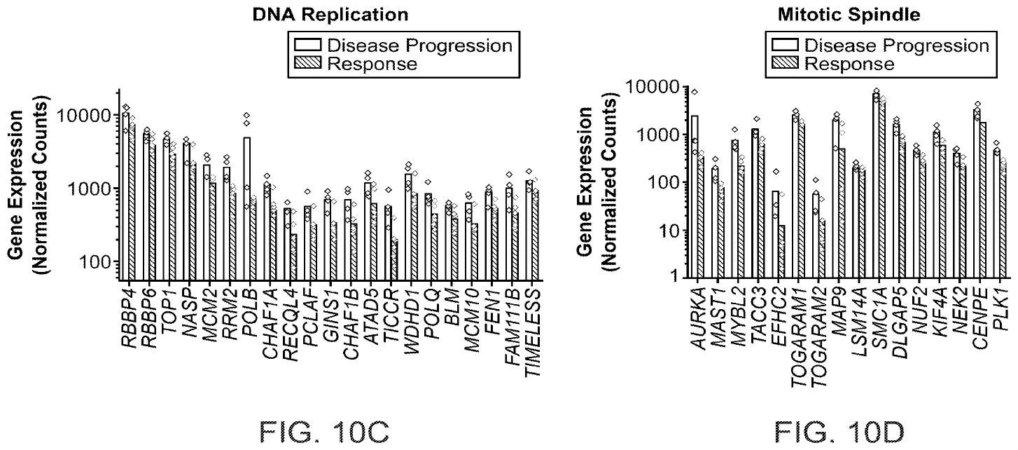

We next assessed the apparent transcriptional activity of the RB1/E2F axis in patient tumors using quantitative gene expression analysis of TAZ responding and non-responding tumors biopsied before and after TAZ treatment using RNA-seq. Pre-treatment TAZ-resistant tumors exhibited significant enrichment of multiple Gene Ontology (GO) terms associated with the cell cycle and in particular with the S and G2/M phases (FIG. 10A). Similarly, post-treatment tumors that progressed on TAZ showed increased gene expression of GO terms associated with mitosis, as compared to TAZ-responsive tumors (FIG. 10B). Indeed, TAZ-resistant tumors exhibited consistently higher expression of S/G2/M-phase-associated genes prior to treatment (FIGS. 10C-10D). These findings suggest that in addition to the mutations affecting the RB1/E2F axis associated with TAZ resistance, additional mutations not captured by MSK-IMPACT targeted gene sequencing and/or epigenetic dysregulation, such as putative silencing of tumor suppressor genes like CDKN1A or CDKN2A, likely contribute to TAZ resistance and the decoupling of RB1/E2F-mediated proliferation and PRC2-regulated differentiation.

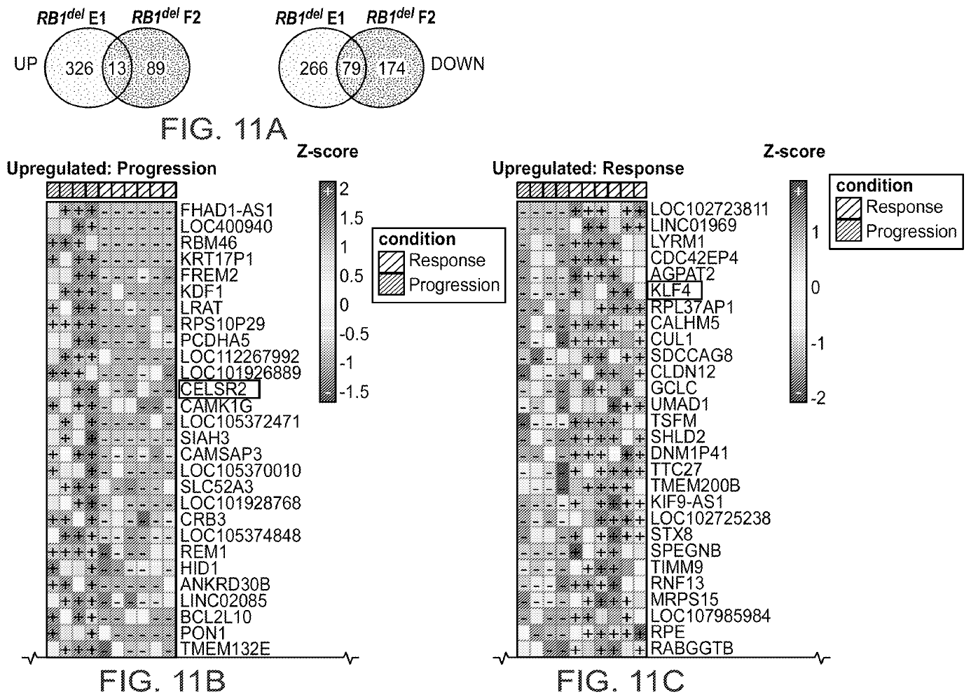

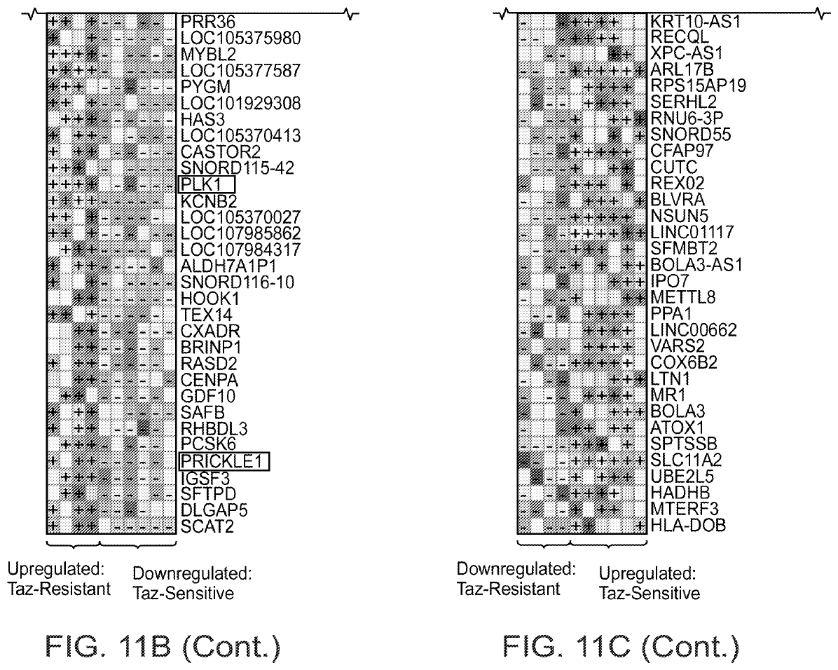

Since TAZ-resistant MRT and ES cell lines and patient tumors show distinct mutations and gene expression changes that converge on the RB1/E2F axis, we inquired whether these perturbations would similarly converge on common prognostic biomarkers of TAZ resistance. Comparative gene expression analysis of untreated RB1^{del} cells versus RB1^{WT}G401 cells showed a small set of consistently and significantly up- and down-regulated genes in two independent clones (FIG. 11A). The most substantially and significantly upregulated gene associated with RB1 loss was PRICKLE1 (FIGS. 3C-3D), which we confirmed to be overexpressed at the protein level in both RB1^{del} clones using Western blotting (FIG. 3E).

In agreement with this, we also found that PRICKLE1 was among the most differentially expressed genes between 10 pre-treatment patient tumors with response and resistance to TAZ, with PRICKLE1 expression being higher in TAZ-resistant tumors (mean normalized reads=10,237 and 300 for resistant and responsive tumors, respectively; Student's t-test p=0.013; FIG. 3F, FIG. 11A). PRICKLE1 can control planar cell polarity (PCP), a key cell differentiation pathway, and has previously been implicated as a prognostic biomarker of poor prognosis in breast cancer (35, 36), acute myeloid leukemia (37), and gastric cancer (38, 39). Several other genes encoding PCP pathway factors were also upregulated in TAZ-resistant tumors compared to TAZ-sensitive tumors (FIG. 11A). We next looked for differentially expressed genes in TAZ-sensitive tumors as potential markers of sensitivity. These included the transcription factor KLF4 which can control the G1/S transition by regulating CDKN1A expression (40, 41) (mean normalized reads=200 and 2,489 for resistant and responsive tumors, respectively; Student's-test p=0.03; FIG. 11B, FIG. 3G). Thus, PRICKLE1 and additional factors controlling PCP and integration of RB1/E2F cell cycle and differentiation are potential prognostic pre-treatment biomarkers to identify clinical TAZ resistance and susceptibility of SMARCB1-deficient tumors.

### Example 5: Synthetic Lethal and Cell Cycle Bypass Epigenetic Combination Strategies Overcome Tazemetostat Resistance

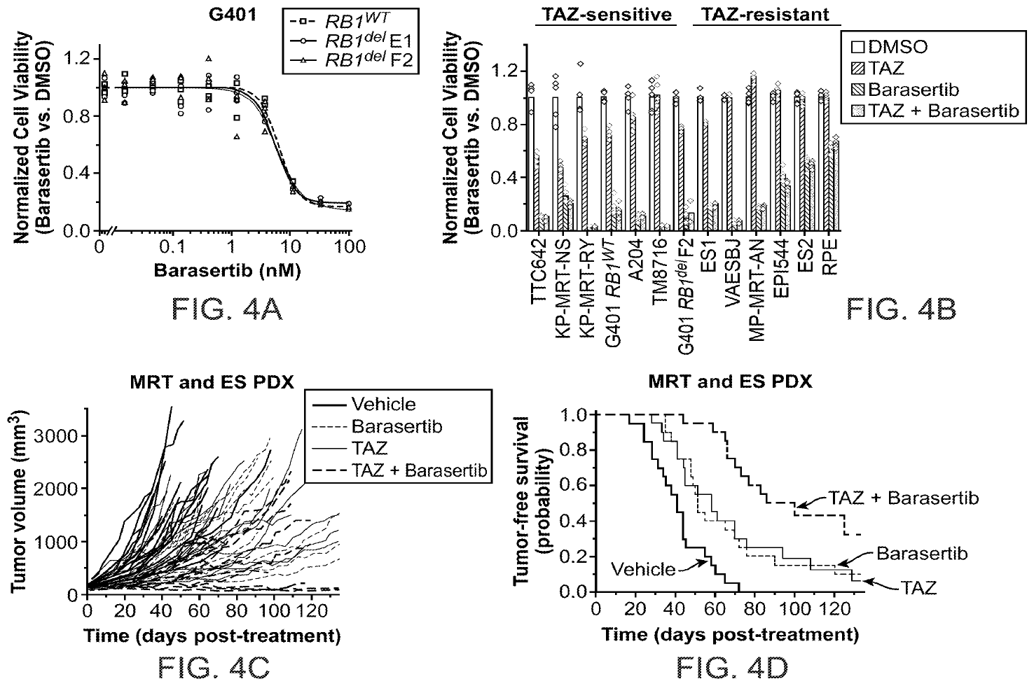

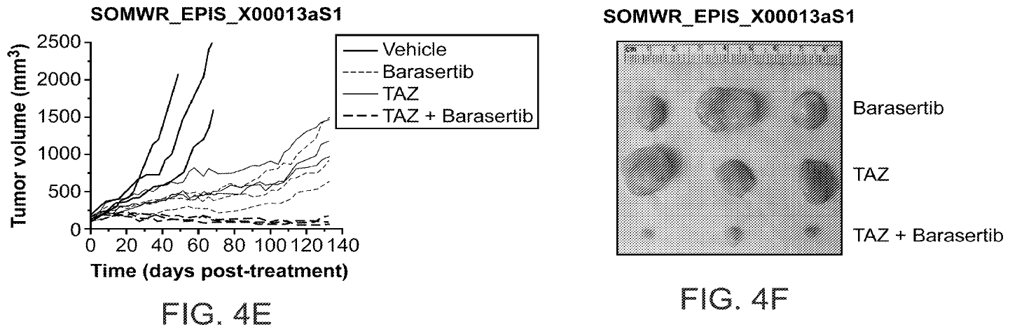

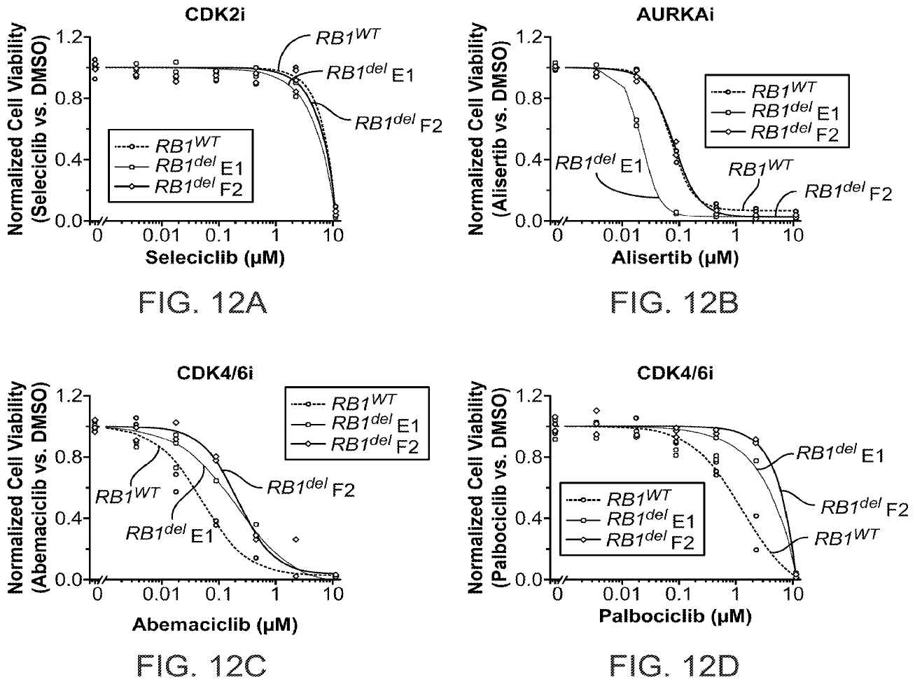

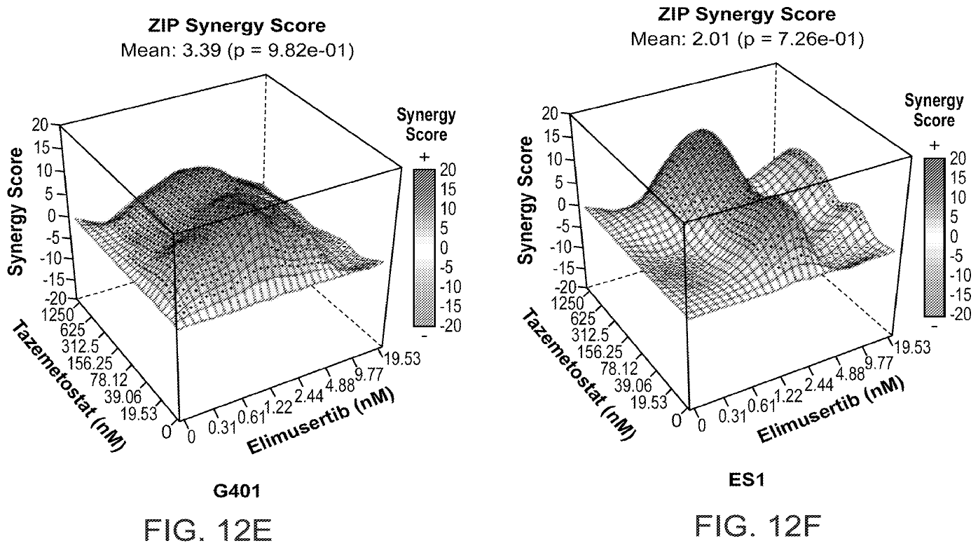

Given that RB1^{del} cells are able to bypass cell cycle arrest at the G1/S checkpoint, we reasoned that inhibiting cell cycle kinases downstream of this checkpoint could overcome TAZ resistance. In particular, cell cycle kinases CDK2 and AURKB, which are downregulated by EZH2 inhibition in TAZ-sensitive cells but persistently expressed in TAZ-resistant cells, may offer especially compelling therapeutic targets to overcome TAZ resistance (FIGS. 2A-2C). Indeed, we found that the CDK2 inhibitor seleciclib (42), as well as the mitotic kinase Aurora A inhibitor alisertib (43), and Aurora B inhibitor barasertib (44), were able to overcome TAZ resistance in RB1^{del} G401 cells (FIG. 4A, FIGS. 12A-12B). We term this combination strategy cell cycle bypass. Consistent with the function of CDK4/6 kinases upstream of RB1/E2F, sensitivity to CDK4/6 inhibitors palbociclib and abemaciclib was reduced by RB1^{del} mutation (FIGS. 12C-12D). In support of the cell cycle bypass strategy for TAZ combination therapy, we observed that patient tumors which progressed on TAZ showed higher expression of AURKB mRNA as compared to those that responded (FIG. 311). Combined with the high sensitivity of G401 cells to barasertib (FIG. 4A; half-maximal effective concentration of 6.5±0.5 nM, 5.5±0.6 nM, and 5.9±0.9 nM for RB1^{WT}, RB1^{del} E1, and F2 clones, respectively), these findings suggest that the cell cycle bypass strategy may effectively overcome TAZ resistance.

We therefore asked whether the combination of TAZ and barasertib would have activity against both TAZ-responsive and TAZ-resistant SMARCB1-deficient MRT and ES cell lines, using RPE cells as a SMARCB1-proficient control. The effects of the combination of TAZ and barasertib on cell viability did not substantially exceed the effect of barasertib alone at the doses tested (FIG. 4B; 200 nM TAZ, 8 nM barasertib). However, nearly all cell lines tested, including those resistant to TAZ monotherapy, showed substantial susceptibility to barasertib, with RPE cells displaying the lowest sensitivity (FIG. 4B).

To test the effects of this combination in vivo, we treated a panel of five patient-derived rhabdoid tumor and epithelioid sarcoma xenografts (PDX) in immunodeficient mice, comparing TAZ and barasertib monotherapies with their combination. Importantly, in contrast to the modest reduction of tumor growth and extension of survival of mice with tumors <1,000 mm³ with TAZ or barasertib alone, PDX mice treated with the combination of TAZ and barasertib showed significant reductions in tumor growth (Vardi U-test p=4.0E-4 and 2.0E-4 for combination versus barasertib or TAZ, respectively; FIG. 4C) (45). In two of the PDX models, this combination led to tumor regressions (FIGS. 4E-4F & 14A). Consistent with this benefit, the combination was also found to significantly increase mean tumor-free animal survival from 65 days (95% confidence interval (CI)=51-78 days) for barasertib and 67 days (95% CI=53-81 days) for TAZ to 98 days (95% CI=84-112 days) for the combination (log-rank test p=3.3E-3 and 5.8E-3 for combination versus barasertib or TAZ, respectively; FIG. 4D). These results indicate that the combination of TAZ with a downstream cell cycle inhibitor such as barasertib can improve response and overcome resistance to TAZ in diverse rhabdoid tumors and epithelioid sarcomas in vivo.

In addition to distinct cell cycle dynamics of TAZ resistance, we observed that regardless of RB1 status, TAZ treatment also caused significant increase in expression of PiggyBac transposable element derived 5 (PGBD5) (FIGS. 2A-2C). PGBD5 is a transposase-derived gene with retained nuclease activity in human cells, which has been implicated as a somatic mutator and inducer of double-strand DNA (dsDNA) breaks in childhood solid tumors (46-48). In rhabdoid tumors in particular, PGBD5 was observed to induce sequence-specific mutations and DNA rearrangements, including somatic deletions of SMARCB1 itself (46). In turn, PGBD5 expression was both necessary and sufficient to confer a cellular dependency on end-joining DNA repair and ATR kinase signaling (47).

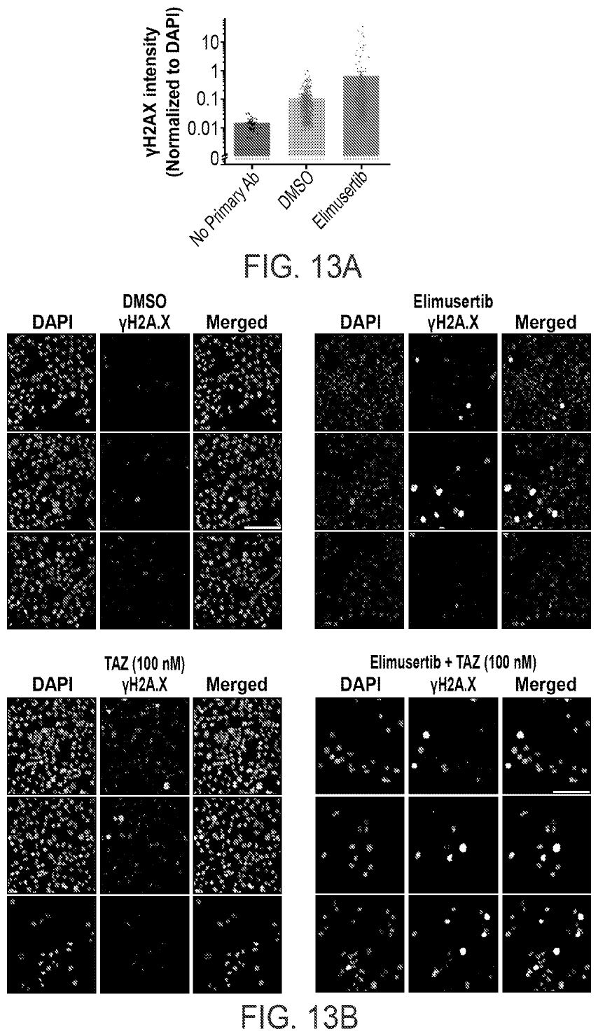

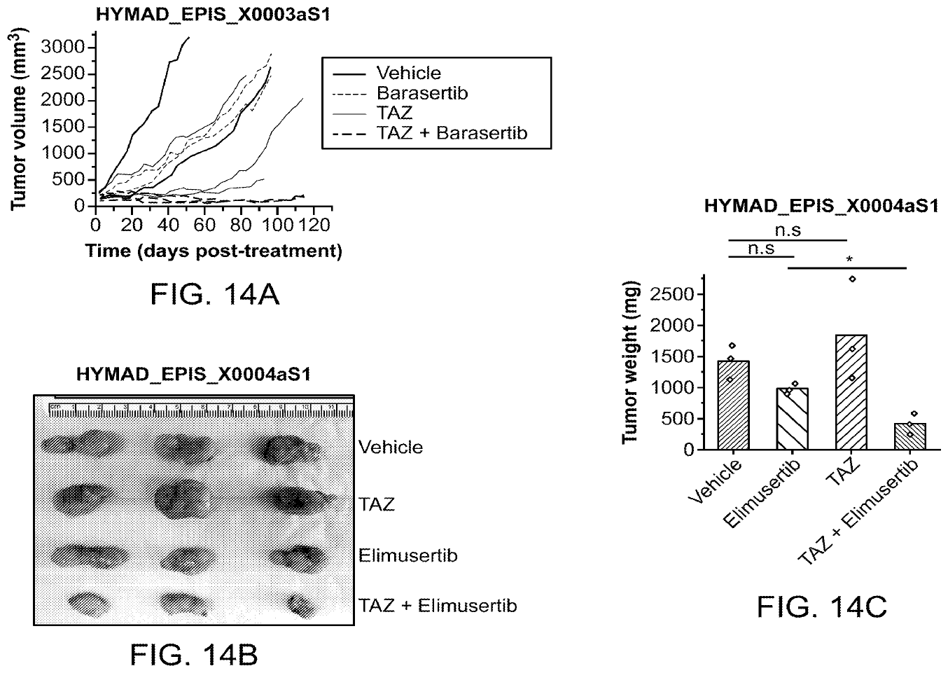

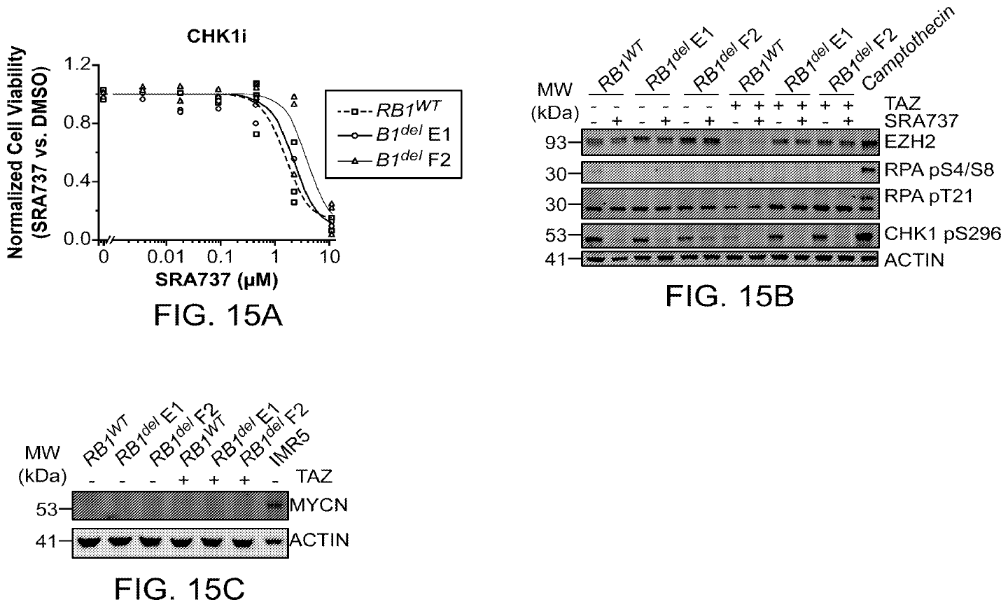

TAZ-induced upregulation of PGBD5 expression suggests that TAZ treatment may potentiate this synthetic lethal dependency. To test this idea, we used the ATR-selective kinase inhibitor elimusertib, which is currently undergoing clinical trials in patients with solid tumors, including patients with PGBD5-expressing tumors such as MRT and ES (Clinical Trials Identifier NCT05071209). We found that elimusertib exhibited low-nanomolar potency against RB1^{WT} and RB1^{del} G401 cells in vitro (half-maximal effective concentration of 17.6±1.6 nM, 19.2±3.8 nM, and 26.7±3.2 nM for RB1^{WT}, RB1^{del} E1 and F2 clones, respectively; FIG. 5A). We also found that the combination of TAZ and elimusertib exerted greater antitumor effects than either drug alone against diverse MRT and ES cell lines (FIG. 5B), exhibiting synergy in a subset of the cell lines (FIGS. 12E-12F). To determine whether the synergistic elimusertib and TAZ combination antitumor effects were due to increased DNA damage, we used confocal immunofluorescence microscopy to quantify γH2AX phosphorylation, a specific marker of dsDNA breaks (49). In agreement with prior studies (47), untreated G401 cells showed dsDNA breaks associated with baseline PGBD5 expression. Consistent with TAZ-mediated induction of PGBD5 expression (FIGS. 2A-2C), we found that TAZ treatment alone significantly increased nuclear γH2AX fluorescence (median normalized level=0.061 versus 0.12, respectively; t-test p=1.7E-8; FIG. 5C), and the combination of TAZ and elimusertib induced additional dose-dependent increases in dsDNA break levels than either drug alone (median normalized level=0.079 versus 0.12 and 0.20, respectively; t-test p=5.1E-3 and 6.5E-4 for 50 and 100 nM TAZ, respectively; FIG. 5D; FIGS. 13A-13B). Targeting TAZ-potentiated and PGBD5-induced DNA damage using the ATR kinase-selective inhibitor elimusertib was specific, because combination of TAZ with the DNA replication repair CHK1 kinase-selective inhibitor SRA737 showed no increased activity as compared to either drug alone (FIGS. 15A-15B). Indeed, TAZ treatment did not induce apparent replication stress, as measured by RPA phosphorylation (FIG. 15B), which was also not potentiated by combined CHK1 inhibition with SRA737, in spite of effective suppression of CHK1 auto-phosphorylation (FIG. 15B). Unlike T-ALL, where EZH2 suppression induces MYCN protein expression and replication stress (50), TAZ treatment of G401 rhabdoid tumor cells failed to increase MYCN protein abundance (FIG. 15C), in spite of significant upregulation of MYCN mRNA (FIGS. 2A-2C).

Encouraged by the potent and specific antitumor activity of synthetic lethal combination TAZ therapy in vitro, we tested the antitumor activity of elimusertib and TAZ combination using a diverse cohort of MRT and ES PDX mice in vivo. The combination of TAZ and elimusertib exceeded the effect of treatment with either drug alone when assessed by tumor measurements (Vardi U-test p=2.0E-2 and 0.19 for combination versus elimusertib or TAZ, respectively; FIG. 5E) and significantly extended tumor-free survival from 51 days (95% CI=42-60 days) for elimusertib and 67 days (95% CI=53-81 days) for TAZ to 99 days (95% CI=74-123 days) for the combination (log-rank test p=5.8E-4 and 3.9E-2 for combination versus elimusertib or TAZ, respectively; FIG. 5F). This was most pronounced for the HYMAD_EPIS_X0004aS1 tumor (FIGS. 5G-5H & 14B-14C), which exhibited a relatively poor response to TAZ monotherapy, when assessed by tumor growth measurements (Vardi U-test p=2.0E-4 for combination versus elimusertib or TAZ; FIG. 5G) and tumor-free survival (log-rank test p=6.2E-3 and 6.3E-5 for combination versus elimusertib or TAZ, respectively; FIG. 511). Thus, the combination of EZH2 and ATR inhibition constitutes a synthetic lethal rational combination strategy to improve TAZ clinical response and overcome resistance.

### Example 6: Overcoming Tazemetostat in Sarcomas

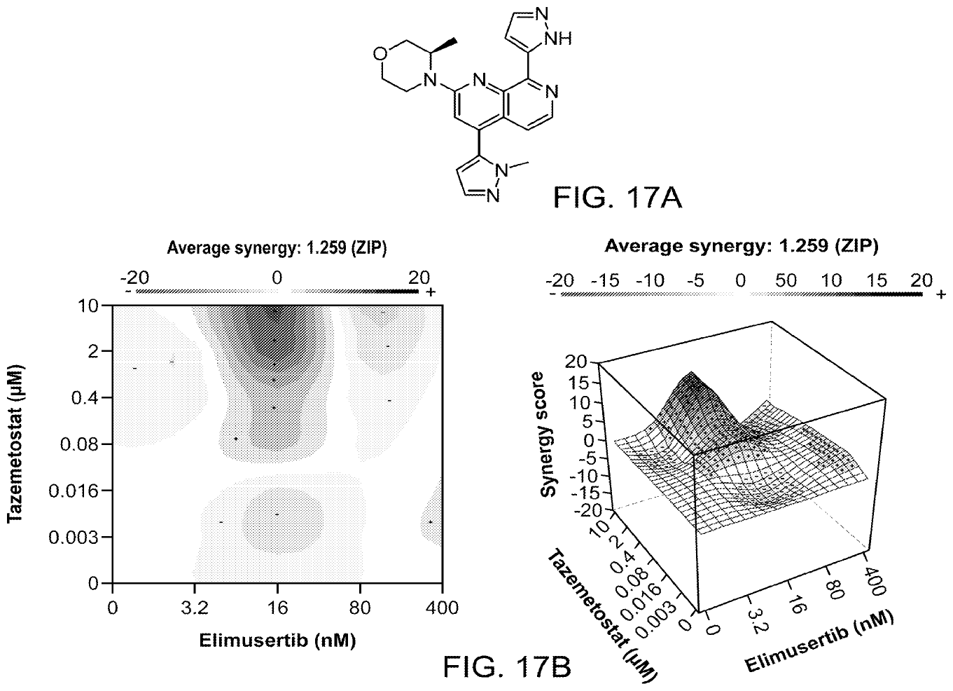

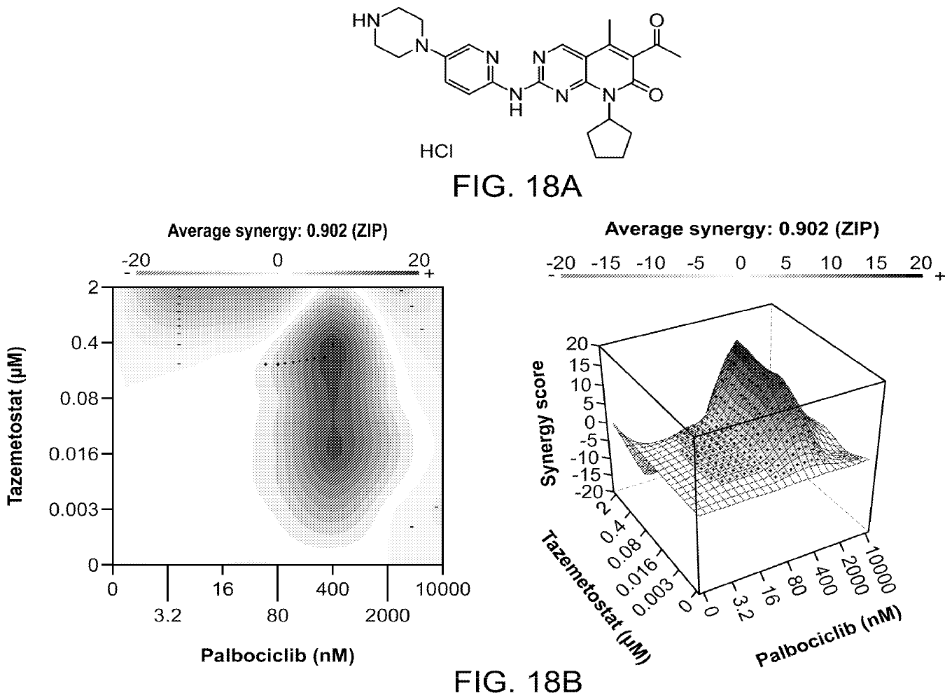

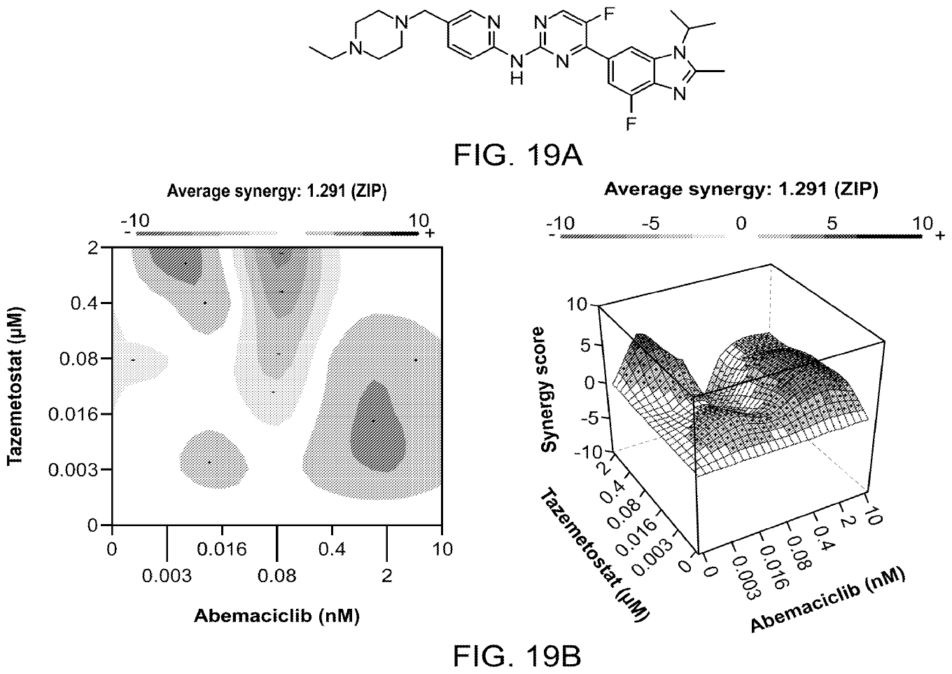

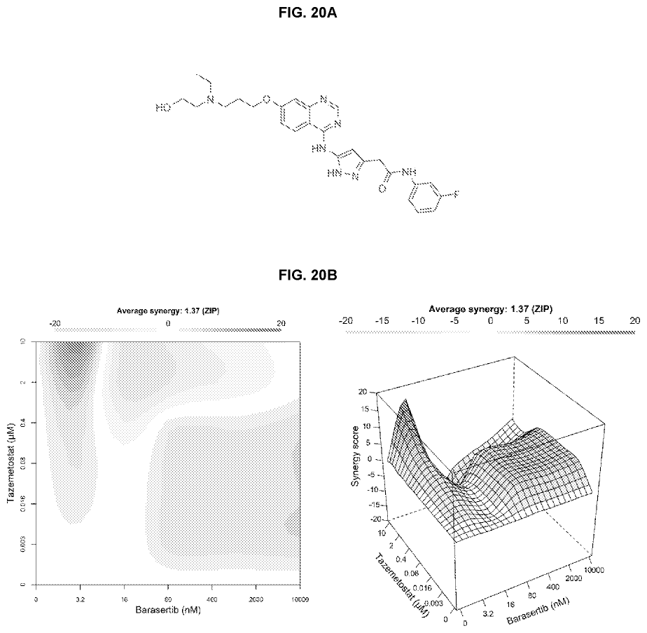

The genetic mechanisms of resistance to tazemetostat were defined through the analysis of patient sarcoma tumors, including matched specimens from patients whose tumors initially regressed and subsequently progressed during a clinical trial with tazemetostat. This included acquired mutation of EZH2, which confers resistance to tazemetostat in EZH2-mutant rhabdoid tumor cells and can be overcome with the allosteric embryonic ectoderm development protein (EED) inhibitor MAK683, but not with the EZH1/2 inhibitor valemetostat. Inactivating mutations of RB1, which cause EZH2-independent resistance, were also observed as evidenced by the effective suppression of H3K27 trimethylation in isogenic pairs of RB1-deficient and proficient rhabdoid tumor cells. Using transcriptomics and cell cycle analysis, it was found that RB1 loss allows cells to escape G1/S arrest caused by tazemetostat-induced upregulation of the cell cycle inhibitor CDKN2A. Thus, an intact RB1/E2F axis is important for therapeutic response to tazemetostat, suggesting a general mechanism for effective epigenetic therapy of this disease and nominating prognostic biomarkers for stratifying future therapy for patients. Translational studies of a panel of diverse rhabdoid and epithelioid sarcoma cell lines and xenografts revealed synergistic combinations with tazemetostat, including epigenetic and synthetic lethal mechanisms. In particular, combination of tazemetostat with the recently developed ATR inhibitor elimusertib (FIGS. 17A-17B) leads to differentiation and apoptosis of rhabdoid tumor cells due to the epigenetic induction of the DNA transposase PGBD5, causing unrepaired dsDNA breaks in both dividing and G1-phase cells in the absence of immediate DNA replication stress. Likewise, CDK4/6 inhibitors in combination with tazemetostat as well as AURKB inhibitors in combination with tazemetostat synergistically induced apoptosis of sarcomas. See FIGS. 18A-18B, FIGS. 19A-19B and FIGS. 20A-20B.

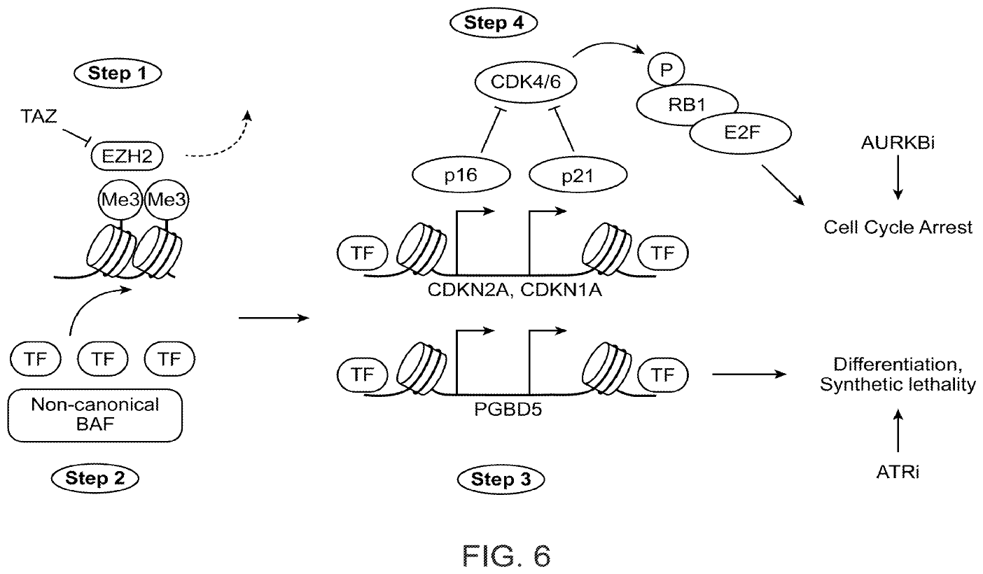

Our studies of ES patients treated with TAZ demonstrate that effective inhibition of PRC2 enzymatic activity is necessary but not sufficient for durable antitumor effects. Using clinical genomics and transcriptomics, combined with functional genetic studies of more than 15 diverse MRT and ES cell lines and patient-derived tumors in vitro and in vivo, a general molecular model for effective epigenetic TAZ therapy is provided herein (FIG. 6). This model places validated RB1 and EZH2 TAZ resistance alleles within the context of a molecular sequence of events required for clinical TAZ response. This model also explains additional mutations associated with TAZ resistance based on the perturbation of each stage of this sequence and provides candidate prognostic biomarkers and therapeutic combination strategies.

First, TAZ must be able to bind and enzymatically inhibit the EZH2 SET domain (FIG. 6; Step 1). This inhibition can be blocked by gatekeeper mutations of the EZH2 drug binding site, as observed in lymphoma cell lines (18), and demonstrated in an epithelioid sarcoma with clinically acquired EZH2^{Y666N} mutation. Such resistance mutations can be overcome by targeting EED, a non-enzymatic PRC2 subunit.

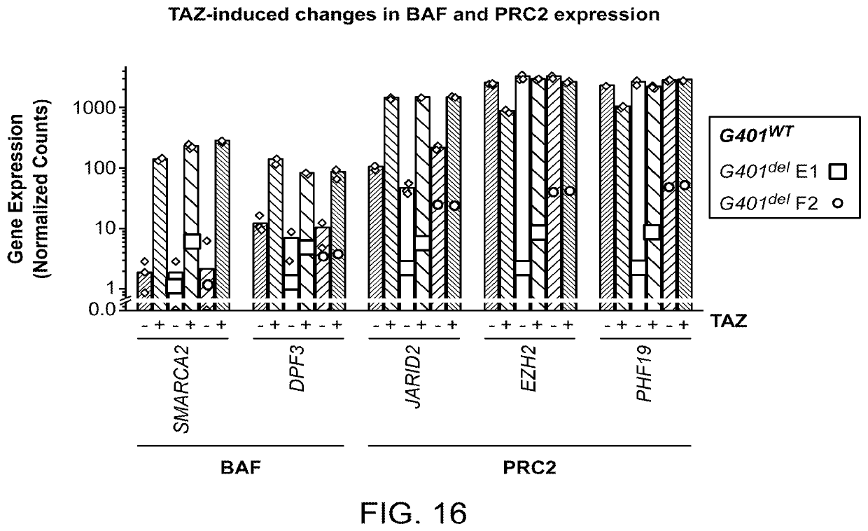

For effective epigenetic TAZ therapy, chromatin remodeling complexes must act on tumor suppressor loci that were aberrantly repressed by PRC2 (FIG. 6; Step 2). The canonical BAF complex is thought to oppose the activity of the Polycomb Repressive Complex (PRC), associated with its chromatin eviction (5, 6). However, the precise mechanism of eviction of TAZ-inhibited PRC2 in SMARCB1-deleted tumors is not fully defined. This may involve TAZ-induced remodeling of BAF and/or PRC2. For example, a recent study of SMARCA4-deficient cell lines found that upregulation of the expression of BAF helicase SMARCA2, which is under PRC2 control in these cells, is necessary for response to EZH2 inhibition (51). In our study, we observed RB1-independent upregulation of the expression of BAF subunits SMARCA2 and DPF3 upon TAZ treatment in G401 cells (FIG. 16). This suggests that TAZ may impact BAF complex assembly as part of its therapeutic mechanism in SMARCB1-deficient tumor cells. However, this also indicates that SMARCA2 re-expression upon EZH2 inhibition is not sufficient to induce cell cycle arrest in the absence of RB1 expression. After assessing TAZ-induced gene expression changes in PRC2 subunits, we also observed RB1-independent TAZ-induced upregulation of PRC2 subunit JARID2, and RB1-dependent downregulation of PHF19, suggesting that PRC2 composition itself may be affected by TAZ treatment (FIG. 16). Additionally, it is unknown whether PRC2 eviction in TAZ-treated cells requires a specific form of the BAF complex, such as the non-canonical SMARCB1-deficient ncBAF or GBAF complex described previously (52, 53).

If the activity of specific BAF subtypes is indeed needed to evict PRC2 from chromatin upon EZH2 inhibition, then genetic perturbation of specific BAF subunits may impact tumor response to TAZ. For example, in our patient cohort, we observed one TAZ-sensitive tumor with a truncation in the canonical BAF-specific subunit ARID1B, while a TAZ-resistant tumor had a missense mutation in PBAF-specific subunit ARID2 (FIG. 1A). We also found ARID1B to be mutated in 2 out of 5 TAZ-responsive cell lines (0 out of 5 TAZ-sensitive cell lines) and ARID2 to be mutated in 2 out of 5 TAZ-resistant cell lines, though also in 1 out of 5 TAZ-sensitive lines (FIG. 3B).

Effective epigenetic TAZ therapy must also upregulate PRC2-repressed tumor suppressor loci (FIG. 6; Step 3). In our patient cohort, one TAZ-resistant tumor had deletions of both CDKN2A and CDKN2B (FIG. 1A), which can inhibit CDK4/6 from phosphorylating RB1 and are known to be de-repressed by TAZ treatment (7). Our genomic analysis of MRT and ES cell lines also showed that 4 out of 5 TAZ-resistant cell lines tested had apparent loss of CDKN2A, with two also having loss of CDKN2B, and one having CDKN1A loss as well (FIG. 3B). These mutations may phenocopy RB1 loss, suggesting that upregulation of these cell cycle inhibitors may be necessary for effective TAZ therapy.

In addition to the preservation of these tumor suppressor loci, transcription factors and coactivators that upregulate their expression must also be intact and expressed for tumor cells to effectively respond to TAZ (FIG. 6; Step 3). In our patient cohort, 2 TAZ-resistant patient tumors had missense mutations in ANKRD11 (FIG. 1A), as did 3 out of 5 TAZ-resistant cell lines. ANKRD11 is a putative tumor suppressor that exhibits loss of heterozygosity in breast cancer (54), and is recurrently mutated in other cancers (55, 56). ANRKD11 can cooperate with p53 to upregulate CDKN1A, and previous reports have suggested possible risk of cancer development in patients with a constitutional loss of ANKRD11 (57, 58). While its importance in SMARCB1-deficient sarcomas is unknown, its association with TAZ resistance suggests potential causality. We note that its proposed tumor suppressor function would require all alleles to be lost or mutated. Consistent with potential functional significance, EPI544 cells harbor multiple mutations in ANKRD11, two with an apparent allele frequency of 1.0 and MP-MRT-AN cells harbor two mutations, both with 0.5 allele frequencies. Interestingly, a recent report described a patient with KBG syndrome, a developmental condition caused by mutation of ANKRD11, who also developed a rhabdoid tumor (58). The co-occurrence of these two exceedingly rare conditions in the same patient is consistent with potential functional involvement of ANKRD11 in rhabdoid tumor development and susceptibility to EZH2 inhibition.

We also identified KLF4 as a putative marker of susceptibility to TAZ in patient tumors (FIG. 3G), and found KLF4 to be mutated in 1 out of 5 TAZ-resistant cell lines (FIG. 3B). Although KLF4 is a transcription factor with both an activating and repressing functions, its role in SMARCB1-deleted tumors is not known. This suggests that KLF4 may regulate the induction of tumor suppressor genes in response to EZH2 inhibition, such as its bona fide target CDKN1A. This may occur through recruitment of BAF to tumor suppressor loci; KLF4 can recruit BAF to target genes to upregulate them (60). Like ANKRD11, KLF4 may thus be a key activator of PRC2-repressed genes (FIG. 5).

Finally, effective TAZ therapy also requires the function of downstream cell cycle effectors of the relevant tumor suppressor loci (FIG. 6; Step 4). As we have demonstrated in this study, loss of RB1 leads to the evasion of TAZ-induced cell cycle arrest, despite effective inhibition of EZH2 activity and sustained transcriptional response to TAZ. A recent genome-wide CRISPR screen also found RB1 as a top mediator of TAZ resistance (15). This is reminiscent of the necessity of intact RB1 for therapeutic response to clinical inhibitors of CDK4/6 (61-63). The transcriptional upregulation of hundreds of genes by TAZ in RB1^{del} tumors, including EMT gene sets (FIGS. 8C & 8F), suggests that these cells are undergoing forced differentiation, even while maintaining proliferation (FIGS. 2G-2H). This upregulation of mesenchymal gene sets is consistent with previous work that has shown that PRC2 inhibition, similar to SMARCB1 re-expression, can drive SMARCB1-deficient tumors into a terminally differentiated, mesenchymal-like state (7, 64, 65), possibly recapitulating the normal developmental trajectory of their cells of origin (64). In this way, RB1 loss and dysregulation of the RB1/E2F axis appear to decouple the regulation of cell fate and identity from its control of cell cycle progression.

In our search for predictive biomarkers of TAZ response, we identified increased expression of the PCP gene PRICKLE1 to be associated with a deficient RB1/E2F axis and TAZ resistance (FIGS. 3C-3F). The molecular mechanism connecting G1/S dysregulation and PRICKLE1 expression is currently unknown, but the PCP pathway is known to be under cell cycle control (66, 67). This is mediated at least in part by PLK1, which we found to be one of the top upregulated genes in TAZ-resistant tumors (FIG. 11B). Likewise, deletion of the Drosophila RB1 homologue Rbf1 results in the upregulation of several PCP genes, including the PRICKLE1 homologue pk (68). This suggests that dysregulation of the RB1/E2F axis may lead to upregulation of PRICKLE1 through dysregulation of normal cell cycle control of PCP.

Finally, our study developed two strategies to circumvent clinical TAZ resistance. First, since dysregulation of the RB1/E2F axis mediates escape from cell cycle arrest at the G1/S checkpoint, we reasoned that cell cycle kinases that function downstream of this checkpoint would remain viable therapeutic targets. This cell cycle bypass strategy is supported by previous work showing that loss of RB1 can sensitize cancer cells to Aurora kinase inhibition through a primed spindle assembly checkpoint (69). RB1^{del} cells remain sensitive to CDK2, AURKA, and AURKB inhibition (FIGS. 4A, 12A-12B). We found that MRT and ES cell lines resistant to TAZ, including those with ANKRD11 and CDKN1A/2A/2B mutations were sensitive to barasertib. We found that the combination of TAZ and barasertib exhibits improved antitumor activity in vivo compared to either drug alone.

Accordingly, the combination therapy methods disclosed herein are useful for treating sarcomas in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Kadoch C, Crabtree GR. Mammalian SWI/SNF chromatin remodeling complexes and cancer: Mechanistic insights gained from human genomics. Sci Adv. 2015; 1(5):e1500447.
2. Versteege I, Sevenet N, Lange J, Rousseau-Merck M F, Ambros P, Handgretinger R, et al. Truncating mutations of hSNF5/INI1 in aggressive paediatric cancer. Nature. 1998; 394(6689):203-6.
3. Kim K H, Kim W, Howard T P, Vazquez F, Tsherniak A, Wu I N, et al. SWI/SNF-mutant cancers depend on catalytic and non-catalytic activity of EZH2. Nat Med. 2015; 21(12):1491-6.
4. McKenna E S, Sansam C G, Cho Y J, Greulich H, Evans J A, Thom C S, et al. Loss of the epigenetic tumor suppressor SNF5 leads to cancer without genomic instability. Mol Cell Biol. 2008; 28(20):6223-33.
5. Wilson B G, Wang X, Shen X, McKenna E S, Lemieux M E, Cho Y J, et al. Epigenetic antagonism between polycomb and SWI/SNF complexes during oncogenic transformation. Cancer Cell. 2010; 18(4):316-28.
6. Kadoch C, Williams R T, Calarco J P, Miller E L, Weber C M, Braun S M, et al. Dynamics of BAF-Polycomb complex opposition on heterochromatin in normal and oncogenic states. Nat Genet. 2017; 49(2):213-22.
7. Knutson S K, Warholic N M, Wigle T J, Klaus C R, Allain C J, Raimondi A, et al. Durable tumor regression in genetically altered malignant rhabdoid tumors by inhibition of methyltransferase EZH2. Proc Natl Acad Sci USA. 2013; 110(19):7922-7.
8. McCabe M T, Ott H M, Ganji G, Korenchuk S, Thompson C, Van Aller G S, et al. EZH2 inhibition as a therapeutic strategy for lymphoma with EZH2-activating mutations. Nature. 2012; 492(7427):108-12.
9. Konze K D, Ma A, Li F, Barsyte-Lovejoy D, Parton T, Macnevin C J, et al. An orally bioavailable chemical probe of the Lysine Methyltransferases EZH2 and EZH1. ACS Chem Biol. 2013; 8(6):1324-34.
10. Li C, Wang Y, Gong Y, Zhang T, Huang J, Tan Z, et al. Finding an easy way to harmonize: a review of advances in clinical research and combination strategies of EZH2 inhibitors. Clin Epigenetics. 2021; 13(1):62.
11. Guerra S, Cichowski K. Targeting Cancer at the Intersection of Signaling and Epigenetics. Annual Review of Cancer Biology. 2019; 3(1):365-84.
12. Baylin S B, Jones P A. Epigenetic Determinants of Cancer. Cold Spring Harb Perspect Biol. 2016; 8(9).
13. Gounder M, Schoffski P, Jones R L, Agulnik M, Cote G M, Villalobos V M, et al. Tazemetostat in advanced epithelioid sarcoma with loss of INI1/SMARCB1: an international, open-label, phase 2 basket study. Lancet Oncol. 2020; 21(11):1423-32.
14. Chi S N, Bourdeaut F, Casanova M, Kilburn L B, Hargrave D R, McCowage G B, et al. Update on phase 1 study of tazemetostat, an enhancer of zeste homolog 2 inhibitor, in pediatric patients with relapsed or refractory integrase interactor 1-negative tumors. Journal of Clinical Oncology. 2022; 40(16_suppl):10040-.
15. Drosos Y, Myers J A, Xu B, Mathias K M, Beane E C, Radko-Juettner S, et al. NSD1 mediates antagonism between SWI/SNF and polycomb complexes and is required for transcriptional activation upon EZH2 inhibition. Mol Cell. 2022; 82(13):2472-89.e8.
16. Cheng D T, Mitchell T N, Zehir A, Shah R H, Benayed R, Syed A, et al. Memorial Sloan Kettering-Integrated Mutation Profiling of Actionable Cancer Targets (MSK-IMPACT): A Hybridization Capture-Based Next-Generation Sequencing Clinical Assay for Solid Tumor Molecular Oncology. J Mol Diagn. 2015; 17(3):251-64.
17. Bracken A P, Pasini D, Capra M, Prosperini E, Colli E, Helin K. EZH2 is downstream of the pRB-E2F pathway, essential for proliferation and amplified in cancer. Embo j. 2003; 22(20):5323-35.
18. Gibaja V, Shen F, Harari J, Korn J, Ruddy D, Saenz-Vash V, et al. Development of secondary mutations in wild-type and mutant EZH2 alleles cooperates to confer resistance to EZH2 inhibitors. Oncogene. 2016; 35(5):558-66.
19. Bisserier M, Wajapeyee N. Mechanisms of resistance to EZH2 inhibitors in diffuse large B-cell lymphomas. Blood. 2018; 131(19):2125-37.
20. Brooun A, Gajiwala K S, Deng Y-L, Liu W, Bolanos B, Bingham P, et al. Polycomb repressive complex 2 structure with inhibitor reveals a mechanism of activation and drug resistance.
21. Honma D, Kanno O, Watanabe J, Kinoshita J, Hirasawa M, Nosaka E, et al. Novel orally bioavailable EZH1/2 dual inhibitors with greater antitumor efficacy than an EZH2 selective inhibitor. Cancer Sci. 2017; 108(10):2069-78.
22. He Y, Selvaraju S, Curtin M L, Jakob C G, Zhu H, Comess K M, et al. The EED protein-protein interaction inhibitor A-395 inactivates the PRC2 complex. Nature Chemical Biology. 2017; 13(4):389-95.
23. Qi W, Zhao K, Gu J, Huang Y, Wang Y, Zhang H, et al. An allosteric PRC2 inhibitor targeting the H3K27me3 binding pocket of EED. Nat Chem Biol. 2017; 13(4):381-8.
24. Tomassi S, Romanelli A, Zwergel C, Valente S, Mai A. Polycomb Repressive Complex 2 Modulation through the Development of EZH2-EED Interaction Inhibitors and EED Binders. Journal of Medicinal Chemistry. 2021; 64(16):11774-97.
25. Coe B P, Thu K L, Aviel-Ronen S, Vucic E A, Gazdar A F, Lam S, et al. Genomic deregulation of the E2F/Rb pathway leads to activation of the oncogene EZH2 in small cell lung cancer. PLoS One. 2013; 8(8):e71670.
26. Kenny C, O'Meara E, Ula M, Hokamp K, O'Sullivan M J. Global Chromatin Changes Resulting from Single—Gene Inactivation—The Role of SMARCB1 in Malignant Rhabdoid Tumor. Cancers (Basel). 2021; 13(11).
27. Kia S K, Gorski M M, Giannakopoulos S, Verrijzer C P. SWI/SNF mediates polycomb eviction and epigenetic reprogramming of the INK4b-ARF-INK4a locus. Mol Cell Biol. 2008; 28(10):3457-64.
28. Kotake Y, Cao R, Viatour P, Sage J, Zhang Y, Xiong Y. pRB family proteins are required for H3K27 trimethylation and Polycomb repression complexes binding to and silencing p161NK4alpha tumor suppressor gene. Genes Dev. 2007; 21(1):49-54.
29. Gallagher D, Voronova A, Zander M A, Cancino G I, Bramall A, Krause M P, et al. Ankrdl 1 is a chromatin regulator involved in autism that is essential for neural development. Dev Cell. 2015; 32(1):31-42.
30. Neilsen P M, Cheney K M, Li C W, Chen J D, Cawrse J E, Schulz R B, et al. Identification of ANKRD11 as a p53 coactivator. J Cell Sci. 2008; 121(Pt 21):3541-52.
31. Noll J E, Jeffery J, Al-Ejeh F, Kumar R, Khanna K K, Callen D F, et al. Mutant p53 drives multinucleation and invasion through a process that is suppressed by ANKRD11. Oncogene. 2012; 31(23):2836-48.
32. Beguelin W, Rivas M A, Calvo Fernindez M T, Teater M, Purwada A, Redmond D, et al. EZH2 enables germinal centre formation through epigenetic silencing of CDKN1A and an Rb-E2F1 feedback loop. Nat Commun. 2017; 8(1):877.
33. Fan T, Jiang S, Chung N, Alikhan A, Ni C, Lee C C, et al. EZH2-dependent suppression of a cellular senescence phenotype in melanoma cells by inhibition of p21/CDKN1A expression. Mol Cancer Res. 2011; 9(4):418-29.
34. Negishi M, Wongpalee S P, Sarkar S, Park J, Lee K Y, Shibata Y, et al. A new lncRNA, APTR, associates with and represses the CDKN1A/p21 promoter by recruiting polycomb proteins. PLoS One. 2014; 9(4):e95216.
35. Daulat A M, Finetti P, Revinski D, Silveira Wagner M, Camoin L, Audebert S, et al. ECT2 associated to PRICKLE1 are poor-prognosis markers in triple-negative breast cancer. Br J Cancer. 2019; 120(9):931-40.
36. Daulat A M, Bertucci F, Audebert S, Serge A, Finetti P, Josselin E, et al. PRICKLE1 Contributes to Cancer Cell Dissemination through Its Interaction with mTORC2. Dev Cell. 2016; 37(4):311-25.
37. Jiang D, He Y, Mo Q, Liu E, Li X, Huang L, et al. PRICKLE1, a Wnt/PCP signaling component, is overexpressed and associated with inferior prognosis in acute myeloid leukemia. J Transl Med. 2021; 19(1):211.
38. Zhou R, Tang Z, Li H, Wang X, Sun Y. PRICKLE1 promotes gastric cancer metastasis by activating mTOR signaling. Am J Transl Res. 2021; 13(5):4266-80.
39. Hu J, Yang Y, Ma Y, Ning Y, Chen G, Liu Y. Proliferation Cycle Transcriptomic Signatures are Strongly associated With Gastric Cancer Patient Survival. Front Cell Dev Biol. 2021; 9:770994.
40. Chen X, Johns D C, Geiman D E, Marban E, Dang D T, Hamlin G, et al. Kruppel-like factor 4 (gut-enriched Kruppel-like factor) inhibits cell proliferation by blocking G1/S progression of the cell cycle. J Biol Chem. 2001; 276(32):30423-8.
41. Chen X, Whitney E M, Gao S Y, Yang V W. Transcriptional profiling of Kruppel-like factor 4 reveals a function in cell cycle regulation and epithelial differentiation. J Mol Biol. 2003; 326(3):665-77.
42. De Azevedo W F, Leclerc S, Meijer L, Havlicek L, Strnad M, Kim S H. Inhibition of cyclin-dependent kinases by purine analogues: crystal structure of human cdk2 complexed with roscovitine. Eur J Biochem. 1997; 243(1-2):518-26.
43. Manfredi M G, Ecsedy J A, Chakravarty A, Silverman L, Zhang M, Hoar K M, et al. Characterization of Alisertib (MLN8237), an investigational small-molecule inhibitor of aurora A kinase using novel in vivo pharmacodynamic assays. Clin Cancer Res. 2011; 17(24):7614-24.
44. Wilkinson R W, Odedra R, Heaton S P, Wedge S R, Keen N J, Crafter C, et al. AZD1152, a selective inhibitor of Aurora B kinase, inhibits human tumor xenograft growth by inducing apoptosis. Clin Cancer Res. 2007; 13(12):3682-8.
45. Vardi Y, Ying Z, Zhang C H. Two-sample tests for growth curves under dependent right censoring. Biometrika. 2001; 88(4):949-60.
46. Henssen A G, Koche R, Zhuang J, Jiang E, Reed C, Eisenberg A, et al. PGBD5 promotes site-specific oncogenic mutations in human tumors. Nat Genet. 2017; 49(7):1005-14.
47. Henssen A G, Reed C, Jiang E, Garcia H D, von Stebut J, MacArthur I C, et al. Therapeutic targeting of PGBD5-induced DNA repair dependency in pediatric solid tumors. Science translational medicine. 2017; 9(414).
48. Henssen A G, Henaff E, Jiang E, Eisenberg A R, Carson J R, Villasante C M, et al. Genomic DNA transposition induced by human PGBD5. Elife. 2015; 4.
49. Mah L J, El-Osta A, Karagiannis T C. gammaH2A X: a sensitive molecular marker of DNA damage and repair. Leukemia. 2010; 24(4):679-86.
50. Le6n T E, Rapoz-D'Silva T, Bertoli C, Rahman S, Magnussen M, Philip B, et al. EZH2-Deficient T-cell Acute Lymphoblastic Leukemia Is Sensitized to CHK1 Inhibition through Enhanced Replication Stress. Cancer Discov. 2020; 10(7):998-1017.
51. Januario T, Ye X, Bainer R, Alicke B, Smith T, Haley B, et al. PRC2-mediated repression of SMARCA2 predicts EZH2 inhibitor activity in SWI/SNF mutant tumors. Proc Natl Acad Sci USA. 2017; 114(46):12249-54.
52. Wang X, Wang S, Troisi E C, Howard T P, Haswell J R, Wolf B K, et al. BRD9 defines a SWI/SNF sub-complex and constitutes a specific vulnerability in malignant rhabdoid tumors. Nat Commun. 2019; 10(1):1881.
53. Michel B C, D'Avino A R, Cassel S H, Mashtalir N, McKenzie Z M, McBride M J, et al. A non-canonical SWI/SNF complex is a synthetic lethal target in cancers driven by BAF complex perturbation. Nat Cell Biol. 2018; 20(12):1410-20.
54. Powell J A, Gardner A E, Bais A J, Hinze S J, Baker E, Whitmore S, et al. Sequencing, transcript identification, and quantitative gene expression profiling in the breast cancer loss of heterozygosity region 16q24.3 reveal three potential tumor-suppressor genes. Genomics. 2002; 80(3):303-10.
55. Vasmatzis G, Johnson S H, Knudson R A, Ketterling R P, Braggio E, Fonseca R, et al. Genome-wide analysis reveals recurrent structural abnormalities of TP63 and other p53-related genes in peripheral T-cell lymphomas. Blood. 2012; 120(11):2280-9.
56. Gebauer N, Kunstner A, Ketzer J, Witte H M, Rausch T, Benes V, et al. Genomic insights into the pathogenesis of Epstein-Barr virus-associated diffuse large B-cell lymphoma by whole-genome and targeted amplicon sequencing. Blood Cancer J. 2021; 11(5):102.
57. Isrie M, Hendriks Y, Gielissen N, Sistermans E A, Willemsen M H, Peeters H, et al. Haploinsufficiency of ANKRD 11 causes mild cognitive impairment, short stature and minor dysmorphisms. Eur J Hum Genet. 20. England2012. p. 131-3.
58. Behnert A, Auber B, Steinemann D, Fruhwald M C, Huisinga C, Hussein K, et al. KBG syndrome patient due to 16q24.3 microdeletion presenting with a paratesticular rhabdoid tumor: Coincidence or cancer predisposition?Am J Med Genet A. 2018; 176(6):1449-54.
59. Zhang W, Geiman D E, Shields J M, Dang D T, Mahatan C S, Kaestner K H, et al. The gut-enriched Kruppel-like factor (Kruppel-like factor 4) mediates the transactivating effect of p53 on the p21WAF1/Cip1 promoter. J Biol Chem. 2000; 275(24):18391-8.
60. Moonen J R, Chappell J, Shi M, Shinohara T, Li D, Mumbach M R, et al. KLF4 recruits SWI/SNF to increase chromatin accessibility and reprogram the endothelial enhancer landscape under laminar shear stress. Nat Commun. 2022; 13(1):4941.
61. Schoninger S F, Blain S W. The Ongoing Search for Biomarkers of CDK4/6 Inhibitor Responsiveness in Breast Cancer. Mol Cancer Ther. 2020; 19(1):3-12.
62. Finn R S, Dering J, Conklin D, Kalous O, Cohen D J, Desai A J, et al. P D 0332991, a selective cyclin D kinase 4/6 inhibitor, preferentially inhibits proliferation of luminal estrogen receptor-positive human breast cancer cell lines in vitro. Breast Cancer Res. 2009; 11(5):R77.
63. Dean J L, Thangavel C, McClendon A K, Reed C A, Knudsen E S. Therapeutic CDK4/6 inhibition in breast cancer: key mechanisms of response and failure. Oncogene. 2010; 29(28):4018-32.
64. Custers L, Khabirova E, Coorens THH, Oliver TRW, Calandrini C, Young M D, et al. Somatic mutations and single-cell transcriptomes reveal the root of malignant rhabdoid tumours. Nat Commun. 2021; 12(1):1407.
65. Nakayama R T, Pulice J L, Valencia A M, McBride M J, McKenzie Z M, Gillespie M A, et al. SMARCB1 is required for widespread BAF complex-mediated activation of enhancers and bivalent promoters. Nat Genet. 2017; 49(11):1613-23.
66. Noatynska A, Tavernier N, Gotta M, Pintard L. Coordinating cell polarity and cell cycle progression: what can we learn from flies and worms?Open Biol. 2013; 3(8):130083.
67. Shrestha R, Little K A, Tamayo J V, Li W, Perlman D H, Devenport D. Mitotic Control of Planar Cell Polarity by Polo-like Kinase 1. Dev Cell. 2015; 33(5):522-34.
68. Payankaulam S, Yeung K, McNeill H, Henry R W, Arnosti D N. Regulation of cell polarity determinants by the Retinoblastoma tumor suppressor protein. Sci Rep. 2016; 6:22879.
69. Gong X, Du J, Parsons S H, Merzoug F F, Webster Y, Iversen P W, et al. Aurora A Kinase Inhibition Is Synthetic Lethal with Loss of the RB1 Tumor Suppressor Gene. Cancer Discov. 2019; 9(2):248-63.
70. Palmer A C, Sorger P K. Combination Cancer Therapy Can Confer Benefit via Patient-to-Patient Variability without Drug Additivity or Synergy. Cell. 2017; 171(7):1678-91.e13.
71. Law L W. Effects of combinations of antileukemic agents on an acute lymphocytic leukemia of mice. Cancer Res. 1952; 12(12):871-8.
72. Frei E, 3rd, Karon M, Levin R H, Freireich E J, Taylor R J, Hananian J, et al. The effectiveness of combinations of antileukemic agents in inducing and maintaining remission in children with acute leukemia. Blood. 1965; 26(5):642-56.
73. Tsuda Y, Iimori M, Nakashima Y, Nakanishi R, Ando K, Ohgaki K, et al. Mitotic slippage and the subsequent cell fates after inhibition of Aurora B during tubulin-binding agent-induced mitotic arrest. Sci Rep. 2017; 7(1):16762.
74. Hauf S, Cole R W, LaTerra S, Zimmer C, Schnapp G, Walter R, et al. The small molecule Hesperadin reveals a role for Aurora B in correcting kinetochore-microtubule attachment and in maintaining the spindle assembly checkpoint. J Cell Biol. 2003; 161(2):281-94.
75. Meerbrey K L, Hu G, Kessler J D, Roarty K, Li M Z, Fang J E, et al. The pINDUCER lentiviral toolkit for inducible RNA interference in vitro and in vivo. Proc Natl Acad Sci USA. 2011; 108(9):3665-70.
76. Eisenhauer E A, Therasse P, Bogaerts J, Schwartz L H, Sargent D, Ford R, et al. New response evaluation criteria in solid tumours: revised RECIST guideline (version 1.1). Eur J Cancer. 2009; 45(2):228-47.
77. Cerami E, Gao J, Dogrusoz U, Gross B E, Sumer S O, Aksoy B A, et al. The cBio cancer genomics portal: an open platform for exploring multidimensional cancer genomics data. Cancer Discov. 2012; 2(5):401-4.
78. Gao J, Aksoy B A, Dogrusoz U, Dresdner G, Gross B, Sumer S O, et al. Integrative analysis of complex cancer genomics and clinical profiles using the cBioPortal. Sci Signal. 2013; 6(269):pl1.
79. Olshen A B, Venkatraman E S, Lucito R, Wigler M. Circular binary segmentation for the analysis of array-based DNA copy number data. Biostatistics. 2004; 5(4):557-72.

1. A method for selecting a sarcoma patient for treatment with tazemetostat comprising:
detecting mRNA or polypeptide expression and/or activity levels of KLF4 in a biological sample obtained from the sarcoma patient that are elevated compared to a control sample obtained from a healthy subject or a predetermined threshold; and
administering to the sarcoma patient an effective amount of tazemetostat.

2. A method for selecting a sarcoma patient for treatment with tazemetostat and an additional therapeutic agent comprising:
(A)
detecting mRNA or polypeptide expression and/or activity levels of one or more of PRICKLE1, PLK1, and CELSR2 in a biological sample obtained from the sarcoma patient that are elevated compared to a control sample obtained from a healthy subject or a predetermined threshold; and
administering to the sarcoma patient an effective amount of tazemetostat and the additional therapeutic agent, wherein the additional therapeutic agent is an ATR inhibitor, an AURK inhibitor or a CDK4/6 inhibitor; or
(B)
detecting mRNA or polypeptide expression and/or activity levels of KLF4 in a biological sample obtained from the sarcoma patient that are reduced compared to a control sample obtained from a healthy subject or a predetermined threshold; and
administering to the sarcoma patient an effective amount of tazemetostat and the additional therapeutic agent, wherein the additional therapeutic agent is an ATR inhibitor, an AURK inhibitor or a CDK4/6 inhibitor.

3. (canceled)

4. The method of claim 2, wherein mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH).

5. The method of claim 2, wherein polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

6. A method for treating sarcoma in a patient in need thereof comprising administering to the patient an effective amount of tazemetostat and an effective amount of a CDK4/6 inhibitor, an AURK inhibitor or an ATR inhibitor.

7. The method of claim 2, wherein the CDK4/6 inhibitor is selected from the group consisting of palbociclib, ribociclib, and abemaciclib.

8. The method of claim 2, wherein the AURK inhibitor is a selective inhibitor of AURKB or a pan AURK inhibitor.

9. The method of claim 8, wherein the AURK inhibitor is selected from the group consisting of tozasertib, SP-96, AT9283, danusertib (PHA-739358), AMG900, cenisertib, SNS-314, barasertib, hesperadin, AZD1152, GSK1070916, CYC116, BI 811283, AZD2811, PHA680632, reversine, CCT129202, CCT137690, S49076, PF-03814735, chiauranib, Alisertib, quercetin, and VX-680.

10. The method of claim 2, wherein the ATR inhibitor is selected from the group consisting of elimusertib (BAY1895344), Schisandrin B, NU6027, dactolisib (NVP-BEZ235), EPT-46464, Torin 2, VE-821, AZ20, M4344 (VX-803), berzosertib (M6620 (VX-970)), and ceralasertib (AZD6738).

11. The method of claim 2, wherein the patient is non-responsive to at least one prior line of cancer therapy.

12. The method of claim 11, wherein the at least one prior line of cancer therapy is chemotherapy or immunotherapy.

13. The method of claim 2, wherein the sarcoma is a rhabdoid sarcoma or an epithelioid sarcoma.

14. The method of claim 2, wherein the sarcoma comprises a deficiency in a BAF chromatin remodeling complex component or SMARCB1.

15. The method of claim 2, wherein the patient comprises an acquired mutation in EZH2, CDKN2A B, CDKN1A, ANKRD11 or RB1.

16. The method of claim 2, wherein the patient exhibits acquired resistance or intrinsic resistance to tazemetostat.

17. The method of claim 2, wherein the patient is a child or an adult.

18. The method of claim 2, wherein the CDK4/6 inhibitor, the AURK inhibitor, or the ATR inhibitor is sequentially, simultaneously, or separately administered with tazemetostat.

19. The method of claim 2, wherein the CDK4/6 inhibitor, the AURK inhibitor, the ATR inhibitor or tazemetostat is administered orally, intravenously, intramuscularly, intraperitoneally, or subcutaneously.

20. (canceled)
